(19) United States
(12) Patent Application Publication
(10) Pub. No.: US 2012/0087916 A1
(43) Pub. Date: Apr. 12, 2012

(54) ANTI-ICAM-1 ANTIBODY, USES AND METHODS

(76) Inventors: Bjorn Frendeus, Landskrona (SE); Niina Veitonmaki, Lund (SE); Titti Martinsson Niskanen, Loddekopinge (SE)

(21) Appl. No.: 13/203,173

(22) PCT Filed: Feb. 13, 2010
(86) PCT No.: PCT/EP2010/001115

§ 371 Date: Nov. 10, 2011

Related U.S. Application Data
(60) Provisional application No. 61/155,325, filed on Feb. 25, 2009.

(30) Foreign Application Priority Data
Feb. 25, 2009 (GB) 0903151.9

(51) Int. Cl. A61K 39/395; A61P 35/00; C07K 16/30

(52) U.S. Cl. 424/133.1; 530/389.7; 424/174.1; 530/387.3; 424/173.1; 424/156.1

(57) ABSTRACT

The present invention relates to an antibody or an antigen-binding fragment thereof with binding specificity for ICAM-1, and to the use thereof in medicine for the treatment of cancers, such as multiple myeloma.

The invention relates to the uses of an antibody or an antigen-binding fragment thereof with binding specificity for ICAM-1, or a variant, fusion or derivative of said antibody or an antigen-binding fragment with binding specificity for ICAM-1, for the treatment of cancer, particularly multiple myeloma. The invention also relates to methods for the administration of such antibodies, fragments, variants, fusion and derivatives thereof.

Multiple myeloma (also referred to as myeloma) is a malignancy of B cells and accounts for 10% to 20% of total haematological malignancies. At present, it is an incurable disease with a median age at diagnosis of 65-70 years, and with very few patients diagnosed below the age of 40. In the United States, 19,920 new cases of multiple myeloma and more than 10,000 deaths are expected in 2008 to be myeloma-related (American Cancer Society, 2008). The disease has a slight male preponderance and is found more frequently in African Americans and less commonly in Asian populations (Kyle & Rajkumar, Blood. 2008 Mar. 15; 111(6):2962-72. Review).

The diagnosis of myeloma carries a grave prognosis, with a median survival of 3 to 4 years with currently available treatments. However, the clinical pattern covers a continuum from indolent forms (e.g., smoldering myeloma) up to high-risk disease with a median survival of only 2 years, even with optimal treatment (Kyle & Rajkumar, Blood. 2008 Mar. 15; 111(6):2962-72. Review.

The typical clinical picture of myeloma is a patient with severe pain due to pathological bone fractures, particularly in the rib cage or vertebral column (Kyle & Rajkumar, 2004, N Engl J Med. 2004 Oct. 28; 351(18):1860-73. Review). Other common features are renal failure, hypercalcemia, bone marrow insufficiency with anemia and thrombocytopenia, and also increased risks of infection and thromboembolic complications such as venous thrombosis and pulmonary embolism. Organ failure is sometimes caused by pathological deposition of fibrillar aggregates of immunoglobulin light chains, called AL-amyloidosis. When present, it typically involves heart and kidneys resulting in severe cardiac arrhythmias and/or failure, and renal malfunction and failure, respectively.

In recent years, substantial progress has been made in understanding the pathogenesis and molecular mechanisms of multiple myeloma. Genetic studies have revealed the occurrence of a vast array of different chromosomal changes, often carrying prognostic relevance, connected with this disease. Briefly, these chromosomal translocations often involve the immunoglobulin (Ig) H locus (14q32.3) and juxtaposes various transforming genes to segments promoted by the Ig enhancer, causing a dysregulated expression and potentially malignant transformation (Hideshima et al. Nat Rev Cancer. 2007. 7(8): 585-598). The therapeutic effect of proteasome inhibition with bortezomib in myeloma was first demonstrated in myeloma cells in vitro, and is probably a result of direct cytotoxicity and of a decrease in the expression of adhesion molecules and various growth, survival and angiogenic factors (Kyle & Rajkumar N Engl J. Med. 2004 Oct. 28; 351(18):1860-73. Review). The transcription factor NFκB, has enhanced activity in myeloma due to proteasomal degradation of its normal regulator protein IκB, and bortezomib reinstates NFκB homeostasis by inhibiting proteasome activity.

The bone marrow microenvironment, consisting of bone osteoclasts, endothelial cells, bone marrow stem cells, as well as extracellular matrix proteins, has a crucial role in multiple myeloma pathogenesis (Hideshima et al., Nat Rev Cancer. 2007. 7(8): 585-598), and provide factors mediating growth, survival and drug resistance of the malignant plasma cells. Various adhesion molecules expressed by the myeloma cells are important for this interaction, for example ICAM-1.

The intercellular adhesion molecule-1 (ICAM-1) is highly expressed and involved in the pathogenesis of multiple types of human malignancies, including myeloma (Huang, et al. Hybridoma. 1993. 12(6): 661-675; Huang et al. Cancer Res. 1995. 55(3): 610-616; Smallshaw et al. Immunother 1997. 2004. 27(6): 419-424; Schmidmaier, Int J Biol Markers. 2006. 21(4): 218-222), melanoma (Wang et al. Int J. Cancer. 2006. 118(4): 932-941; Johnson et al., Immunobiology. 1988. 178(3): 275-284), lung cancer (Grothey et al. Br J Cancer. 1998. 77(5): 801-807), gastric cancer (Maruo et al. Int J. Cancer. 2002. 100(4): 486-490), bladder cancer (Roche et al. Thromb Haemost. 2003. 89(6): 1089-1097), breast cancer (Rosette C, et al. Carcinogenesis. 2005. 26(5): 943-950), prostate cancer (Aalinkeel R et al. Cancer Res 2004. 64(15): 5311-21), and lymphoma (Horst et al. Leukemia. 1991. 5(10): 848-853). Increased ICAM-1 expression is associated with development of drug-induced resistance (Schmidmaier et al. Int J Biol Markers. 2006. 21(4): 218-222), tumour cell aggressiveness (Miele et al., Exp Cell Res 214 (1), 231 1994) and poor prognosis Dowlati et al., Clin Cancer Res 14 (5), 1407 (2008).

Standard treatment for myeloma in younger patients (i.e. less than 65 years of age) has consisted of conditioning with vincristine-adriamycin-dexamethasone followed by high-dose melphalan with autologous stem cell support. During the previous decade, this regime was shown to prolong median survival by approximately 1 year, in spite of achieving complete remission in the bone marrow in only a minority of patients (Harousseau J L. Hematology Am Soc Hematol Educ Program. 2008; 2008:306-12). Due to the risks attached to high-dose treatment, elderly patients have primarily been offered treatment with low-dose melphalan combined with prednisone.

In recent years, other therapies have been approved for the treatment of relapsed myeloma. These new drugs comprise the proteasome-inhibitor bortezomib (Velcade®), and the “immunomodulatory” drugs, thalidomide and lenalidomide (Revlimid®), and constitute a significant progress in treatment options. The overall response rate for relapsed myeloma patients with these drugs is usually around 30%, but generally higher when the drug is combined with intermittent dexamethasone. Based on these findings the new drugs, combined with dexamethasone and/or chemotherapy, are now in clinical trials as first line treatment for myeloma (http://clinicaltrials.gov/ct2/search) with promising preliminary results (American Society of Hematology, Dec. 6-9, 2008).

Although bortezomib, lenalidomide and thalidomide have shown a survival benefit in comparison to traditional therapy in relapsed myeloma patients (Rajkumar Blood. 2005. 106(13): 4050-4053; Richardson et al. Blood. 2006. 108(10): 3458-3464; Richardson et al. N Engl J. Med. 2005. 352(24): 2487-2498; Singhal et al. N Engl J. Med. 1999. 341(21): 1565-1571), the goal of increasing long-term survival or a cure has yet not been reached. Furthermore, the newer drugs also have serious side effects, for example increased risks of thromboembolism, neuropathy and immune and bone marrow suppression, limiting their use in a significant number of patients.

Despite recent advances in the development of novel therapies the actual benefit of these drugs on patient survival and quality of life are modest, warranting development of novel more effective and complimentary therapeutics to combat multiple myeloma. Accordingly, new potential targets and drugs for myeloma therapy are required.

The above problems are addressed by the present invention.

In a first aspect, the invention provides the use of an antibody or an antigen-binding fragment thereof with binding specificity for ICAM-1, or a variant, fusion or derivative of said antibody or an antigen-binding fragment, or a fusion of a said variant or derivative thereof with binding specificity for ICAM-1,
wherein the antibody or antigen-binding fragment thereof comprises one or more of the following amino acid sequences:

[SEQ ID NO: 1]
FSNAWMSWVRQAPG;
and/or
[SEQ ID NO: 2]
AFIWYDGSNKYYADSVKGR;
and/or
[SEQ ID NO: 3]
ARYSGWYFDY;
and/or
[SEQ ID NO: 4]
CTGSSSNIGAGYDVH;
and/or
[SEQ ID NO: 5]
DNNNRPS;
and/or
[SEQ ID NO: 6]
CQSYDSSLSAWL.

in the manufacture of a medicament for the treatment of cancer, the treatment comprising the step of administering to a patient in need thereof an effective amount of the antibody, antigen-binding fragment, variant, fusion or derivative thereof.

In a second aspect, the invention provides an antibody or an antigen-binding fragment thereof with binding specificity for ICAM-1, or a variant, fusion or derivative of said antibody or an antigen-binding fragment, or a fusion of a said variant or derivative thereof with binding specificity for ICAM-1,
wherein the antibody or antigen-binding fragment thereof comprises one or more of the following amino acid sequences:

[SEQ ID NO: 1]
FSNAWMSWVRQAPG;
and/or
[SEQ ID NO: 2]
AFIWYDGSNKYYADSVKGR;
and/or
[SEQ ID NO: 3]
ARYSGWYFDY;
and/or
[SEQ ID NO: 4]
CTGSSSNIGAGYDVH;
and/or
[SEQ ID NO: 5]
DNNNRPS;
and/or
[SEQ ID NO: 6]
CQSYDSSLSAWL.

for use in the treatment of cancer, the treatment comprising the step of administering to a patient in need thereof an effective amount of the antibody, antigen-binding fragment, variant, fusion or derivative thereof.

In a third aspect, the invention provides a method for treating cancer in an individual, the method comprising the step of administering to a patient in need thereof an effective amount of:
an antibody or an antigen-binding fragment thereof with binding specificity for ICAM-1,
or a variant, fusion or derivative of said antibody or an antigen-binding fragment, or a fusion of a said variant or derivative thereof with binding specificity for ICAM-1,
wherein the antibody or antigen-binding fragment thereof comprises one or more of the following amino acid sequences:

[SEQ ID NO: 1]
FSNAWMSWVRQAPG;
and/or
[SEQ ID NO: 2]
AFIWYDGSNKYYADSVKGR;
and/or
[SEQ ID NO: 3]
ARYSGWYFDY;
and/or
[SEQ ID NO: 4]
CTGSSSNIGAGYDVH;
and/or
[SEQ ID NO: 5]
DNNNRPS;
and/or
[SEQ ID NO: 6]
CQSYDSSLSAWL.

As discussed in the accompanying Examples, the present inventors have surprisingly discovered that cancers (such as multiple myeloma) can be effectively treated using an antibody or an antigen-binding fragment with binding specificity for ICAM-1 and which comprise one or more of SEQ ID NO:1-6 (or variants, fusions or derivatives of said antibody or an antigen-binding fragment). It will be appreciated by persons skilled in the art that the above amino acid sequences of SEQ ID NOS:1 to 6 represent complementarity determining regions (CDRs).

Accordingly, the present invention provides new approaches and/or regimens for treating cancers such as multiple myeloma.

Multiple myeloma is a malignant, clonal disease originating from transformed plasma cells. A distinctive feature of the disease is that the malignant cells secrete monoclonal immunoglobulin (Ig), either in the form of IgG- or IgA-type (rarely IgD or IgE), or only light-chains (κ or λ), or both. A finding of monoclonal immunoglobulin (in blood or urine) is not mandatory, however, and in 5% to 10% of cases the myeloma is classified as “nonsecretory.”

The typical clinical presentation of myeloma is a patient with severe pain caused by pathological bone fractures, particularly in the rib cage or vertebral column. Other common features include renal failure caused by free light chains interfering with glomerular or tubular functions and hypercalcemia secondary to enhanced osteoclast activity in bone marrow. Bone marrow insufficiency with anemia and thrombocytopenia is common because of extensive bone marrow infiltration by the plasma cell clone and paramalignant refractoriness to hematopoietic growth factors. Organ failure is sometimes caused by pathological deposition of fibrillar aggregates of immunoglobulin light chains, called amyloid light-chain amyloidosis. Organ failure typically involves heart and kidneys, resulting in severe cardiac arrhythmias or failure and nephrotic syndrome, respectively. Bleeding diathesis is also common in amyloid light-chain amyloidosis. In summary, the fatalities of this cancer type are often a result of bone marrow depression, renal failure, immune incompetence, thromboembolism, or amyloidosis of vital organs.

By ‘treatment’ we include both therapeutic and prophylactic treatment of a subject/patient. The term ‘prophylactic’ is used to encompass the use of a polypeptide or composition described herein which either prevents or reduces the likelihood of the occurrence or development of cancer (such as multiple myeloma) in a patient or subject.

A ‘therapeutically effective amount’, or ‘effective amount’, or ‘therapeutically effective’, as used herein, refers to that amount which provides a therapeutic effect for a given condition and administration regimen. This is a predetermined quantity of active material calculated to produce a desired therapeutic effect in association with the required additive and diluent, i.e. a carrier or administration vehicle. Further, it is intended to mean an amount sufficient to reduce or prevent a clinically significant deficit in the activity, function and response of the host. Alternatively, a therapeutically effective amount is sufficient to cause an improvement in a clinically significant condition in a host.

In a preferred embodiment, the uses of the first and second aspect of the invention and the method of the third aspect of the invention, comprise the step of administering to a patient in need thereof an amount of between about 0.02 mg/kg to 20 mg/kg of the antibody, antigen-binding fragment, variant, fusion or derivative thereof.

The accompanying Examples demonstrate that an exemplary antibody of the invention (termed “BI-AB”) has significant in vivo and in vitro activity against multiple myeloma.

Furthermore, the accompanying Examples demonstrate that BI-AB has significant in vivo anti-myeloma efficacy when administered in doses of 0.016 mg/kg, 0.16 mg/kg and 1.6 mg/kg (see, for example, FIG. 2) and displays a comparable or improved activity compared to administration of far higher dosages (such as 20 mg/kg) of Rituximab®.

The antibody as defined herein is therefore particularly advantageous as it can be administered in far lower dosages than Rituximab®. It will be readily appreciated that it is desirable to administer low dosages of antibody to a patient whilst still retaining a therapeutic effect—doing so decreases the cost of the patient treatment, reduces the number and extent of patient side-effects (resulting in less hospitalisation) and infusion cytotoxicity on the patient.

In a particularly preferred embodiment, the amount of the antibody, antigen-binding fragment, variant, fusion or derivative administered to a patient is approximately between: 0.02 mg/kg to 0.10 mg/kg; or 0.10 mg to 0.20 mg/kg; or 0.20 mg to 0.30 mg/kg; or 0.30 mg to 0.40 mg/kg; or 0.40 mg to 0.50 mg/kg; or 0.50 mg to 0.60 mg/kg; or 0.60 mg to 0.70 mg/kg; or 0.70 mg to 0.80 mg/kg; or 0.80 mg to 0.90 mg/kg; or 0.90 mg to 1.00 mg/kg; or 1.00 mg to 1.10 mg/kg; or 1.10 mg to 1.20 mg/kg; or 1.20 mg to 1.30 mg/kg; or 1.30 mg to 1.40 mg/kg; or 1.40 mg to 1.50 mg/kg; or 1.50 mg to 1.60 mg/kg; or 1.60 mg to 1.70 mg/kg; or 1.70 mg to 1.80 mg/kg; or 1.80 mg to 1.90 mg/kg; or 1.90 mg to 2.00 mg/kg; or 2.00 mg/kg to 2.10 mg/kg; or 2.10 mg to 2.20 mg/kg; or 2.20 mg to 2.30 mg/kg; or 2.30 mg to 2.40 mg/kg; or 2.40 mg to 2.50 mg/kg; or 2.50 mg to 2.60 mg/kg; or 2.60 mg to 2.70 mg/kg; or 2.70 mg to 2.80 mg/kg; or 2.80 mg to 2.90 mg/kg; or 2.90 mg to 3.00 mg/kg; or 3.00 mg to 3.10 mg/kg; or 3.10 mg to 3.20 mg/kg; or 3.20 mg to 3.30 mg/kg; or 3.30 mg to 3.40 mg/kg; or 3.40 mg to 3.50 mg/kg; or 3.50 mg to 3.60 mg/kg; or 3.60 mg to 3.70 mg/kg; or 3.70 mg to 3.80 mg/kg; or 3.80 mg to 3.90 mg/kg; or 3.90 mg to 4.00 mg/kg; or 4.00 mg to 4.10 mg/kg; or 4.10 mg to 4.20 mg/kg; or 4.20 mg to 4.30 mg/kg; or 4.30 mg to 4.40 mg/kg; or 4.40 mg to 4.50 mg/kg; or 4.50 mg to 4.60 mg/kg; or 4.60 mg to 4.70 mg/kg; or 4.70 mg to 4.80 mg/kg; or 4.80 mg to 4.90 mg/kg; or 4.90 mg to 5.00 mg/kg; or 5.00 mg/kg to 6.00 mg/kg; or 6.00 mg to 7.00 mg/kg; or 7.00 mg to 8.00 mg/kg; or 8.00 mg to 9.00 mg/kg; or 9.00 mg to 10.00 mg/kg; or 10.00 mg to 11.00 mg/kg; or 11.00 mg to 12.00 mg/kg; or 12.00 mg to 13.00 mg/kg; or 13.00 mg to 14.00 mg/kg; or 14.00 mg to 15.00 mg/kg; or 15.00 mg to 16.00 mg/kg; or 16.00 mg to 17.00 mg/kg; or 17.00 mg to 18.00 mg/kg; or 18.00 mg to 19.00 mg/kg; or 19.00 mg to 20.00 mg/kg.

In an alternative embodiment, the amount of the antibody, antigen-binding fragment, variant, fusion or derivative administered to a patient is approximately: 0.02 mg/kg; or 0.03 mg/kg; or 0.04 mg/kg; or 0.05 mg/kg; or 0.06 mg/kg; or 0.07 mg/kg; or 0.08 mg/kg; or 0.09 mg/kg; or 0.10 mg/kg; or 0.15 mg/kg; or 0.20 mg/kg; or 0.25 mg/kg; or 0.30 mg/kg; or 0.35 mg/kg; or 0.40 mg/kg; or 0.45 mg/kg; or 0.50 mg/kg; or 0.60 mg/kg; or 0.70 mg/kg; or 0.80 mg/kg; or 0.90 mg/kg; or 1.00 mg/kg; or 1.10 mg/kg; or 1.20 mg/kg; or 1.30 mg/kg; or 1.40 mg/kg; or 1.50 mg/kg; or 1.60 mg/kg; or 1.70 mg/kg; or 1.80 mg/kg; or 1.90 mg/kg; or 2.00 mg/kg; or 2.10 mg/kg; or 2.20 mg/kg; or 2.30 mg/kg; or 2.40 mg/kg; or 2.50 mg/kg; or 2.60 mg/kg; or 2.70 mg/kg; or 2.80 mg/kg; or 2.90 mg/kg; or 3.00 mg/kg; or 3.10 mg/kg; or 3.20 mg/kg; or 3.30 mg/kg; or 3.40 mg/kg; or 3.50 mg/kg; or 3.60 mg/kg; or 3.70 mg/kg; or 3.80 mg/kg; or 3.90 mg/kg; or 4.00 mg/kg; or 4.10 mg/kg; or 4.20 mg/kg; or 4.30 mg/kg; or 4.40 mg/kg; or 4.50 mg/kg; or 4.60 mg/kg; or 4.70 mg/kg; or 4.80 mg/kg; or 4.90 mg/kg; or 5.00 mg/kg; or 6.00 mg/kg; or 7.00 mg/kg; or 8.00 mg/kg; or 9.00 mg/kg; or 10.00 mg/kg; or 11.00 mg/kg; or 12.00 mg/kg; or 13.00 mg/kg; or 14.00 mg/kg; or 15.00 mg/kg; or 16.00 mg/kg; or 17.00 mg/kg; or 18.00 mg/kg; or 19.00 mg/kg; or 20.00 mg/kg.

In a preferred embodiment of the first, second and third aspect of the invention, there is provided a use or method wherein the effective amount of the antibody, antigen-binding fragment, variant, fusion or derivative thereof is selected from:
between 0.02 mg/kg and 0.03 mg/kg; or
between 0.02 mg/kg and 0.30 mg/kg; or
between 0.02 mg/kg and 0.10 mg/kg; or
between 0.10 mg/kg and 0.30 mg/kg.

More preferably, the effective amount of the antibody, antigen-binding fragment, variant, fusion or derivative thereof is selected from: 0.016 mg/kg; or 0.02 mg/kg; or 0.03 mg/kg; or 0.10 mg/kg; or 0.16 mg/kg; or 0.30 mg/kg.

As will be appreciated by those skilled in the art, treatment with antibodies can offer therapeutic advantages with low toxicity in their ability to target cancerous cells and sparing surrounding tissues. The tolerability may reflect the dynamic actions of immunoglobulins, utilizing physiological mechanisms such as natural killer (NK)-cell mediated cell-death or directly inducing apoptosis rather than necrosis of tumour cells.

As is appreciated by those skilled in the art, the precise amount of a compound may vary depending on its specific activity. Suitable dosage amounts may contain a predetermined quantity of active composition calculated to produce the desired therapeutic effect in association with the required diluent. In the methods and use for manufacture of compositions of the invention, a therapeutically effective amount of the active component is provided. A therapeutically effective amount can be determined by the ordinary skilled medical or veterinary worker based on patient characteristics, such as age, weight, sex, condition, complications, other diseases, etc., as is well known in the art.

In a fourth aspect, the invention provides the use of an antibody or an antigen-binding fragment thereof with binding specificity for ICAM-1, or a variant, fusion or derivative of said antibody or an antigen-binding fragment, or a fusion of a said variant or derivative thereof, with binding specificity for ICAM-1,
wherein the antibody or antigen-binding fragment thereof comprises one or more of the following amino acid sequences:

[SEQ ID NO: 1]
FSNAWMSWVRQAPG;
and/or
[SEQ ID NO: 2]
AFIWYDGSNKYYADSVKGR;
and/or
[SEQ ID NO: 3]
ARYSGWYFDY;
and/or
[SEQ ID NO: 4]
CTGSSSNIGAGYDVH;
and/or
[SEQ ID NO: 5]
DNNNRPS;
and/or
[SEQ ID NO: 6]
CQSYDSSLSAWL.

in the manufacture of a medicament for the treatment of cancer, the treatment comprising the step of administering to a patient in need thereof an effective amount of the antibody, antigen-binding fragment, variant, fusion or derivative thereof, in a single dosage at a frequency of once per week or less.

In a fifth aspect, the invention provides, an antibody or an antigen-binding fragment thereof with binding specificity for ICAM-1, or a variant, fusion or derivative of said antibody or an antigen-binding fragment, or a fusion of a said variant or derivative thereof with binding specificity for ICAM-1
wherein the antibody or antigen-binding fragment thereof comprises one or more of the following amino acid sequences:

[SEQ ID NO: 1]
FSNAWMSWVRQAPG;
and/or
[SEQ ID NO: 2]
AFIWYDGSNKYYADSVKGR;
and/or
[SEQ ID NO: 3]
ARYSGWYFDY;
and/or
[SEQ ID NO: 4]
CTGSSSNIGAGYDVH;
and/or
[SEQ ID NO: 5]
DNNNRPS;
and/or
[SEQ ID NO: 6]
CQSYDSSLSAWL.

for use in the treatment of cancer, the treatment comprising the step of administering to a patient in need thereof an effective amount of the antibody, antigen-binding fragment, variant, fusion or derivative thereof, in a single dosage at a frequency of once per week or less.

In a sixth aspect, the invention provides a method for treating cancer in an individual, the method comprising the step of administering to a patient in need thereof a single dosage at a frequency of once per week or less of an effective amount of:
an antibody or an antigen-binding fragment thereof with binding specificity for ICAM-1,
or a variant, fusion or derivative of said antibody or an antigen-binding fragment, or a fusion of a said variant or derivative thereof, with binding specificity for ICAM-1,
wherein the antibody or antigen-binding fragment thereof comprises one or more of the following amino acid sequences:

[SEQ ID NO: 1]
FSNAWMSWVRQAPG;
and/or
[SEQ ID NO: 2]
AFIWYDGSNKYYADSVKGR;
and/or
[SEQ ID NO: 3]
ARYSGWYFDY;
and/or
[SEQ ID NO: 4]
CTGSSSNIGAGYDVH;
and/or
[SEQ ID NO: 5]
DNNNRPS;
and/or
[SEQ ID NO: 6]
CQSYDSSLSAWL.

In the methods and uses of the fourth, fifth and sixth aspects of the invention, the antibody, antigen-binding fragment, variant, fusion or derivative, is administered to a patient in need thereof in a single dose.

It is particularly preferred that the fourth, fifth and sixth aspects of the invention provide a use or method wherein the effective amount of the antibody, antigen-binding fragment, variant, fusion or derivative thereof is administered in a single dosage at a frequency selected from:
once in eight days; or
once in nine days; or
once in ten days; or
once in eleven days; or
once in twelve days; or
once in thirteen days; or
once in fourteen days; or
once in twenty-one days.

The effective amount of the antibody, antigen-binding fragment, variant, fusion or derivative administered in the fourth, fifth and sixth aspects of the invention is as defined above in relation to the first, second and third aspects of the invention.

It will be understood that “a single dose” means that the entire dose of antibody, antigen-binding fragment, variant, fusion or derivative is administered to the patient at one time, for example in a continuous, single and defined treatment without a substantial or significant break or disruption in that treatment, or any division of the dosage over a significant or substantial period.

The single dose of antibody, antigen-binding fragment, variant, fusion or derivative may be administered by one or more administration route, such that the patient receives the single dose in the continuous, single and defined treatment period.

For example, the single dose may be administered by a single injection, or single intravenous infusion, a single subcutaneous injection, or by a single procedure using other routes of administration, as discussed below. Alternatively, the single dose may be administered to the patient by two or more injections given simultaneously or sequentially to deliver the entire dose to the patient in the continuous, single and defined treatment period; by two or more intravenous infusions given simultaneously or sequentially to deliver the entire dose to the patient in the continuous, single and defined treatment; or by multiple procedures using other routes of administration as discussed below.

Alternatively, the single dose to be administered to the patient can be delivered by a combination of routes to deliver the entire dose to the patient in the continuous, single and defined treatment.

Preferably, the dose administered in the uses and methods of the fourth, fifth and sixth aspects of the invention is as defined above in relation to the first, second and third aspects of the invention.

In the methods and uses of the fourth, fifth and sixth aspects of the invention, the antibody, antigen-binding fragment, variant, fusion or derivative, is administered to a patient in need thereof at a frequency of once per week (i.e. once in seven days), or administered less frequently than once per week.

For example, the antibody, antigen-binding fragment, variant, fusion or derivative, may be administered to a patient in need thereof at a frequency of: once in eight days; or once in nine days; or once in ten days; or once in eleven days; or once in twelve days; or once in thirteen days; or once in fourteen days; or once in twenty-one days; or once in 28 days.

Preferably, the treatment frequency is repeated over an overall treatment period of: one month; or two months; or three months; or four months; or five months; or six months; or seven months; or eight months; or nine months; or ten months; or eleven months; or one year; or two years; or three years; or four years; or five years or more.

It will be appreciated that an advantage of the methods and uses of the fourth, fifth and sixth aspects of the invention will be the reduction in the frequency and/or extent of treatment procedures to the patient. That will reduce the overall cost of patient treatment (because, for example, less professional time is required by medical staff), and reduce discomfort to the patient (because, for example, administration will be performed less often and/or less frequently).

In a seventh aspect, the invention provides the use of an antibody or an antigen-binding fragment thereof with binding specificity for ICAM-1, or a variant, fusion or derivative of said antibody or an antigen-binding fragment, or a fusion of a said variant or derivative thereof, with binding specificity for ICAM-1,
wherein the antibody or antigen-binding fragment thereof comprises one or more of the following amino acid sequences:

[SEQ ID NO: 1]
FSNAWMSWVRQAPG;
and/or
[SEQ ID NO: 2]
AFIWYDGSNKYYADSVKGR;
and/or
[SEQ ID NO: 3]
ARYSGWYFDY;
and/or
[SEQ ID NO: 4]
CTGSSSNIGAGYDVH;
and/or
[SEQ ID NO: 5]
DNNNRPS;
and/or
[SEQ ID NO: 6]
CQSYDSSLSAWL.

in the manufacture of a medicament for the treatment of cancer, the treatment comprising the step of administering to a patient in need thereof an effective amount of the antibody, antigen-binding fragment, variant, fusion or derivative thereof in a single dosage at a frequency of once per week or less, wherein the effective amount of the antibody, antigen-binding fragment, variant, fusion or derivative thereof is between about 0.02 mg/kg to 2 mg/kg of the antibody, antigen-binding fragment, variant, fusion or derivative thereof.

In an eighth aspect, the invention provides an antibody or an antigen-binding fragment thereof with binding specificity for ICAM-1, or a variant, fusion or derivative of said antibody or an antigen-binding fragment, or a fusion of a said variant or derivative thereof, with binding specificity for ICAM-1,
wherein the antibody or antigen-binding fragment thereof comprises one or more of the following amino acid sequences:

[SEQ ID NO: 1]
FSNAWMSWVRQAPG;
and/or
[SEQ ID NO: 2]
AFIWYDGSNKYYADSVKGR;
and/or
[SEQ ID NO: 3]
ARYSGWYFDY;
and/or
[SEQ ID NO: 4]
CTGSSSNIGAGYDVH;
and/or
[SEQ ID NO: 5]
DNNNRPS;
and/or
[SEQ ID NO: 6]
CQSYDSSLSAWL.

for use in the treatment of cancer, the treatment comprising the step of administering to a patient in need thereof an effective amount of the antibody, antigen-binding fragment, variant, fusion or derivative thereof in a single dosage at a frequency of once per week or less, wherein the effective amount of the antibody, antigen-binding fragment, variant, fusion or derivative thereof is between about 0.02 mg/kg to 2 mg/kg of the antibody, antigen-binding fragment, variant, fusion or derivative thereof.

In a ninth aspect, the invention provides a method for treating cancer in an individual, the method comprising the step of administering to a patient in need thereof a single dosage at a frequency of once per week or less of an effective amount of:
an antibody or an antigen-binding fragment thereof with binding specificity for ICAM-1,
or a variant, fusion or derivative of said antibody or an antigen-binding fragment, or a fusion of a said variant or derivative thereof, with binding specificity for ICAM-1,
wherein the effective amount of the antibody, antigen-binding fragment, variant, fusion or derivative thereof is between about 0.02 mg/kg to 2 mg/kg of the antibody, antigen-binding fragment, variant, fusion or derivative thereof,
and wherein the antibody or antigen-binding fragment thereof comprises one or more of the following amino acid sequences:

[SEQ ID NO: 1]
FSNAWMSWVRQAPG;
and/or
[SEQ ID NO: 2]
AFIWYDGSNKYYADSVKGR;
and/or
[SEQ ID NO: 3]
ARYSGWYFDY;
and/or
[SEQ ID NO: 4]
CTGSSSNIGAGYDVH;
and/or
[SEQ ID NO: 5]
DNNNRPS;
and/or
[SEQ ID NO: 6]
CQSYDSSLSAWL.

Preferably, in the uses and methods of the seventh, eighth and ninth aspects of the invention, the effective amount of the antibody, antigen-binding fragment, variant, fusion or derivative is as described above in relation to the first, second or third aspects of the invention, and is administered in single dosage at a frequency as described above in relation to the fourth, fifth and sixth aspects of the invention.

Thus, in a particularly preferred embodiment of the seventh, eighth and ninth aspect of the invention, the amount of the antibody, antigen-binding fragment, variant, fusion or derivative administered to a patient is approximately between: 0.02 mg/kg to 0.10 mg/kg; or 0.10 mg to 0.20 mg/kg; or 0.20 mg to 0.30 mg/kg; or 0.30 mg to 0.40 mg/kg; or 0.40 mg to 0.50 mg/kg; or 0.50 mg to 0.60 mg/kg; or 0.60 mg to 0.70 mg/kg; or 0.70 mg to 0.80 mg/kg; or 0.80 mg to 0.90 mg/kg; or 0.90 mg to 1.00 mg/kg; or 1.00 mg to 1.10 mg/kg; or 1.10 mg to 1.20 mg/kg; or 1.20 mg to 1.30 mg/kg; or 1.30 mg to 1.40 mg/kg; or 1.40 mg to 1.50 mg/kg; or 1.50 mg to 1.60 mg/kg; or 1.60 mg to 1.70 mg/kg; or 1.70 mg to 1.80 mg/kg; or 1.80 mg to 1.90 mg/kg; or 1.90 mg to 2.00 mg/kg; or 2.00 mg/kg to 2.10 mg/kg; or 2.10 mg to 2.20 mg/kg; or 2.20 mg to 2.30 mg/kg; or 2.30 mg to 2.40 mg/kg; or 2.40 mg to 2.50 mg/kg; or 2.50 mg to 2.60 mg/kg; or 2.60 mg to 2.70 mg/kg; or 2.70 mg to 2.80 mg/kg; or 2.80 mg to 2.90 mg/kg; or 2.90 mg to 3.00 mg/kg; or 3.00 mg to 3.10 mg/kg; or 3.10 mg to 3.20 mg/kg; or 3.20 mg to 3.30 mg/kg; or 3.30 mg to 3.40 mg/kg; or 3.40 mg to 3.50 mg/kg; or 3.50 mg to 3.60 mg/kg; or 3.60 mg to 3.70 mg/kg; or 3.70 mg to 3.80 mg/kg; or 3.80 mg to 3.90 mg/kg; or 3.90 mg to 4.00 mg/kg; or 4.00 mg to 4.10 mg/kg; or 4.10 mg to 4.20 mg/kg; or 4.20 mg to 4.30 mg/kg; or 4.30 mg to 4.40 mg/kg; or 4.40 mg to 4.50 mg/kg; or 4.50 mg to 4.60 mg/kg; or 4.60 mg to 4.70 mg/kg; or 4.70 mg to 4.80 mg/kg; or 4.80 mg to 4.90 mg/kg; or 4.90 mg to 5.00 mg/kg; or 5.00 mg/kg to 6.00 mg/kg; or 6.00 mg to 7.00 mg/kg; or 7.00 mg to 8.00 mg/kg; or 8.00 mg to 9.00 mg/kg; or 9.00 mg to 10.00 mg/kg; or 10.00 mg to 11.00 mg/kg; or 11.00 mg to 12.00 mg/kg; or 12.00 mg to 13.00 mg/kg; or 13.00 mg to 14.00 mg/kg; or 14.00 mg to 15.00 mg/kg; or 15.00 mg to 16.00 mg/kg; or 16.00 mg to 17.00 mg/kg; or 17.00 mg to 18.00 mg/kg; or 18.00 mg to 19.00 mg/kg; or 19.00 mg to 20.00 mg/kg.

In an alternative embodiment of the seventh, eighth and ninth aspect of the invention, the amount of the antibody, antigen-binding fragment, variant, fusion or derivative administered to a patient is approximately: 0.02 mg/kg; or 0.03 mg/kg; or 0.04 mg/kg; or 0.05 mg/kg; or 0.06 mg/kg; or 0.07 mg/kg; or 0.08 mg/kg; or 0.09 mg/kg; or 0.10 mg/kg; or 0.15 mg/kg; or 0.20 mg/kg; or 0.25 mg/kg; or 0.30 mg/kg; or 0.35 mg/kg; or 0.40 mg/kg; or 0.45 mg/kg; or 0.50 mg/kg; or 0.60 mg/kg; or 0.70 mg/kg; or 0.80 mg/kg; or 0.90 mg/kg; or 1.00 mg/kg; or 1.10 mg/kg; or 1.20 mg/kg; or 1.30 mg/kg; or 1.40 mg/kg; or 1.50 mg/kg; or 1.60 mg/kg; or 1.70 mg/kg; or 1.80 mg/kg; or 1.90 mg/kg; or 2.00 mg/kg; or 2.10 mg/kg; or 2.20 mg/kg; or 2.30 mg/kg; or 2.40 mg/kg; or 2.50 mg/kg; or 2.60 mg/kg; or 2.70 mg/kg; or 2.80 mg/kg; or 2.90 mg/kg; or 3.00 mg/kg; or 3.10 mg/kg; or 3.20 mg/kg; or 3.30 mg/kg; or 3.40 mg/kg; or 3.50 mg/kg; or 3.60 mg/kg; or 3.70 mg/kg; or 3.80 mg/kg; or 3.90 mg/kg; or 4.00 mg/kg; or 4.10 mg/kg; or 4.20 mg/kg; or 4.30 mg/kg; or 4.40 mg/kg; or 4.50 mg/kg; or 4.60 mg/kg; or 4.70 mg/kg; or 4.80 mg/kg; or 4.90 mg/kg; or 5.00 mg/kg; or 6.00 mg/kg; or 7.00 mg/kg; or 8.00 mg/kg; or 9.00 mg/kg; or 10.00 mg/kg; or 11.00 mg/kg; or 12.00 mg/kg; or 13.00 mg/kg; or 14.00 mg/kg; or 15.00 mg/kg; or 16.00 mg/kg; or 17.00 mg/kg; or 18.00 mg/kg; or 19.00 mg/kg; or 20.00 mg/kg.

In the methods and uses of the of the seventh, eighth and ninth aspect of the invention, the antibody, antigen-binding fragment, variant, fusion or derivative, is administered to a patient in need thereof in a single dose.

The single dose of antibody, antigen-binding fragment, variant, fusion or derivative may be administered by one or more administration route, such that the patient receives the single dose in the continuous, single and defined treatment period.

For example, the single dose may be administered by a single injection, or single intravenous infusion, a single subcutaneous injection, or by a single procedure using other routes of administration, as discussed below. Alternatively, the single dose may be administered to the patient by two or more injections given simultaneously or sequentially to deliver the entire dose to the patient in the continuous, single and defined treatment period; by two or more intravenous infusions given simultaneously or sequentially to deliver the entire dose to the patient in the continuous, single and defined treatment; or by multiple procedures using other routes of administration as discussed below.

Alternatively, the single dose to be administered to the patient can be delivered by a combination of routes to deliver the entire dose to the patient in the continuous, single and defined treatment.

In the methods and uses of the of the seventh, eighth and ninth aspect of the invention, the antibody, antigen-binding fragment, variant, fusion or derivative, is administered to a patient in need thereof at a frequency of once per week (i.e. once in seven days), or administered less frequently than once per week. For example, the antibody, antigen-binding fragment, variant, fusion or derivative, may be administered to a patient in need thereof at a frequency of: once in eight days; or once in nine days; or once in ten days; or once in eleven days; or once in twelve days; or once in thirteen days; or once in fourteen days; or once in twenty-one days; or once in 28 days.

Preferably, the treatment frequency is repeated over an overall treatment period of: one month; or two months; or three months; or four months; or five months; or six months; or seven months; or eight months; or nine months; or ten months; or eleven months; or one year; or two years; or three years; or four years; or five years or more.

It will be appreciated that an advantage of the methods and uses of the seventh, eighth and ninth aspect of the invention will be the reduction in the frequency and/or extent of treatment procedures to the patient. That will reduce the overall cost of patient treatment (because, for example, less professional time is required by medical staff), and reduce discomfort to the patient (because, for example, administration will be performed less often and/or less frequently).

Thus, in a preferred embodiment of the seventh, eighth and ninth aspect of the invention, there is provided a use or method wherein the effective amount of the antibody, antigen-binding fragment, variant, fusion or derivative thereof and administration frequency is selected from:
between 0.02 mg/kg and 0.03 mg/kg, administered at a frequency of once in seven days; or
between 0.02 mg/kg and 0.03 mg/kg, administered at a frequency of once in eight days; or
between 0.02 mg/kg and 0.03 mg/kg, administered at a frequency of once in nine days; or
between 0.02 mg/kg and 0.03 mg/kg, administered at a frequency of once in ten days; or
between 0.02 mg/kg and 0.03 mg/kg, administered at a frequency of once in eleven days; or
between 0.02 mg/kg and 0.03 mg/kg, administered at a frequency of once in twelve days; or
between 0.02 mg/kg and 0.03 mg/kg, administered at a frequency of once in fourteen days; or
between 0.02 mg/kg and 0.03 mg/kg, administered at a frequency of once in twenty-one days.

In another preferred embodiment of the seventh, eighth and ninth aspect of the invention, there is provided a use or method wherein the effective amount of the antibody, antigen-binding fragment, variant, fusion or derivative thereof and administration frequency is selected from:
between 0.02 mg/kg and 0.30 mg/kg, administered at a frequency of once in seven days; or
between 0.02 mg/kg and 0.30 mg/kg, administered at a frequency of once in eight days; or
between 0.02 mg/kg and 0.30 mg/kg, administered at a frequency of once in nine days; or
between 0.02 mg/kg and 0.30 mg/kg, administered at a frequency of once in ten days; or
between 0.02 mg/kg and 0.30 mg/kg, administered at a frequency of once in eleven days; or
between 0.02 mg/kg and 0.30 mg/kg, administered at a frequency of once in twelve days; or
between 0.02 mg/kg and 0.30 mg/kg, administered at a frequency of once in fourteen days; or
between 0.02 mg/kg and 0.30 mg/kg, administered at a frequency of once in twenty-one days.

In another preferred embodiment of the seventh, eighth and ninth aspect of the invention, there is provided a use or method wherein the effective amount of the antibody, antigen-binding fragment, variant, fusion or derivative thereof and administration frequency is selected from:
between 0.02 mg/kg and 0.10 mg/kg, administered at a frequency of once in seven days; or
between 0.02 mg/kg and 0.10 mg/kg, administered at a frequency of once in eight days; or
between 0.02 mg/kg and 0.10 mg/kg, administered at a frequency of once in nine days; or
between 0.02 mg/kg and 0.10 mg/kg, administered at a frequency of once in ten days; or
between 0.02 mg/kg and 0.10 mg/kg, administered at a frequency of once in eleven days; or
between 0.02 mg/kg and 0.10 mg/kg, administered at a frequency of once in twelve days; or
between 0.02 mg/kg and 0.10 mg/kg, administered at a frequency of once in fourteen days; or
between 0.02 mg/kg and 0.10 mg/kg, administered at a frequency of once in twenty-one days.

In another preferred embodiment of the seventh, eighth and ninth aspect of the invention, there is provided a use or method wherein the effective amount of the antibody, antigen-binding fragment, variant, fusion or derivative thereof and administration frequency is selected from:
between 0.10 mg/kg and 0.30 mg/kg, administered at a frequency of once in seven days; or
between 0.10 mg/kg and 0.30 mg/kg, administered at a frequency of once in eight days; or
between 0.10 mg/kg and 0.30 mg/kg, administered at a frequency of once in nine days; or
between 0.10 mg/kg and 0.30 mg/kg, administered at a frequency of once in ten days; or
between 0.10 mg/kg and 0.30 mg/kg, administered at a frequency of once in eleven days; or
between 0.10 mg/kg and 0.30 mg/kg, administered at a frequency of once in twelve days; or
between 0.10 mg/kg and 0.30 mg/kg, administered at a frequency of once in fourteen days; or
between 0.10 mg/kg and 0.30 mg/kg, administered at a frequency of once in twenty-one days.

In another preferred embodiment of the seventh, eighth and ninth aspect of the invention, there is provided a use or method wherein the effective amount of the antibody, antigen-binding fragment, variant, fusion or derivative thereof and administration frequency is selected from:
0.016 mg/kg, administered at a frequency of once in seven days; or
0.016 mg/kg, administered at a frequency of once in eight days; or
0.016 mg/kg, administered at a frequency of once in nine days; or
0.016 mg/kg, administered at a frequency of once in ten days; or
0.016 mg/kg, administered at a frequency of once in eleven days; or
0.016 mg/kg, administered at a frequency of once in twelve days; or
0.016 mg/kg, administered at a frequency of once in fourteen days; or
0.016 mg/kg, administered at a frequency of once in twenty-one days.

In another preferred embodiment of the seventh, eighth and ninth aspect of the invention, there is provided a use or method wherein the effective amount of the antibody, antigen-binding fragment, variant, fusion or derivative thereof and administration frequency is selected from:
0.02 mg/kg, administered at a frequency of once in seven days; or
0.02 mg/kg, administered at a frequency of once in eight days; or
0.02 mg/kg, administered at a frequency of once in nine days; or
0.02 mg/kg, administered at a frequency of once in ten days; or
0.02 mg/kg, administered at a frequency of once in eleven days; or
0.02 mg/kg, administered at a frequency of once in twelve days; or
0.02 mg/kg, administered at a frequency of once in fourteen days; or
0.02 mg/kg, administered at a frequency of once in twenty-one days.

In another preferred embodiment of the seventh, eighth and ninth aspect of the invention, there is provided a use or method wherein the effective amount of the antibody, antigen-binding fragment, variant, fusion or derivative thereof and administration frequency is selected from:
0.03 mg/kg, administered at a frequency of once in seven days; or
0.03 mg/kg, administered at a frequency of once in eight days; or
0.03 mg/kg, administered at a frequency of once in nine days; or
0.03 mg/kg, administered at a frequency of once in ten days; or
0.03 mg/kg, administered at a frequency of once in eleven days; or
0.03 mg/kg, administered at a frequency of once in twelve days; or
0.03 mg/kg, administered at a frequency of once in fourteen days; or
0.03 mg/kg, administered at a frequency of once in twenty-one days.

In another preferred embodiment of the seventh, eighth and ninth aspect of the invention, there is provided a use or method wherein the effective amount of the antibody, antigen-binding fragment, variant, fusion or derivative thereof and administration frequency is selected from:
0.10 mg/kg, administered at a frequency of once in seven days; or
0.10 mg/kg, administered at a frequency of once in eight days; or
0.10 mg/kg, administered at a frequency of once in nine days; or
0.10 mg/kg, administered at a frequency of once in ten days; or
0.10 mg/kg, administered at a frequency of once in eleven days; or
0.10 mg/kg, administered at a frequency of once in twelve days; or
0.10 mg/kg, administered at a frequency of once in fourteen days; or
0.10 mg/kg, administered at a frequency of once in twenty-one days.

In another preferred embodiment of the seventh, eighth and ninth aspect of the invention, there is provided a use or method wherein the effective amount of the antibody, antigen-binding fragment, variant, fusion or derivative thereof and administration frequency is selected from:
0.30 mg/kg, administered at a frequency of once in seven days; or
0.30 mg/kg, administered at a frequency of once in eight days; or
0.30 mg/kg, administered at a frequency of once in nine days; or
0.30 mg/kg, administered at a frequency of once in ten days; or
0.30 mg/kg, administered at a frequency of once in eleven days; or
0.30 mg/kg, administered at a frequency of once in twelve days; or
0.30 mg/kg, administered at a frequency of once in fourteen days; or
0.30 mg/kg, administered at a frequency of once in twenty-one days.

As discussed above, ICAM-1 plays a key role in the onset and progression of multiple myeloma. In addition, it is now known that multiple myeloma patients that have developed resistance to chemotherapy have increased (myeloma cell) expression of ICAM-1 (Schmidmaier et al., Int J Biol Markers 21 (4), 218 (2006)) and that ICAM-1-mediated adhesion of myeloma cells to bone marrow stroma can increase survival of myeloma cells and induce primary multidrug resistance (Schmidmaier et al., Int J Biol Markers 21 (4), 218 (2006)). Those conditions can therefore be effectively treated, inhibited, reduced and/or prevented using the antibody and/or antigen-binding fragment and/or variant, fusion or derivative as defined herein.

In addition to its role in myeloma cell survival, ICAM-1-mediated adhesion appears to play a role in invasion and metastasis of tumour cells. ICAM-1 and its counter receptor LFA-1 both participate in homing of multiple myeloma cells to the bone marrow (Hideshima et al., Cancer 7 (8), 585 (2007); Rosette et al., Carcinogenesis 26 (5), 943 (2005)), and mediate adhesion of multiple myeloma cells to the bone marrow or to the stroma. Inhibition of ICAM-1 by small molecules, antibodies or siRNA in non-clinical studies has been shown to block cancer cells invasion and to decrease cancer cell metastases (Rosette et al., Carcinogenesis 26 (5), 943 (2005); Miele et al., Exp Cell Res 214 (1), 231 (1994); Huang et al., Carcinogenesis 25 (10), 1925 (2004)). In addition to increased cell surface expression of ICAM-1, cancer patients have elevated soluble ICAM-1 (sICAM-1) in the circulation (Gearing & Newman, Immunol Today 14 (10), 506 (1993); Sanchez-Rovira et al., Eur J Cancer 34 (3), 394 (1998); van de Stolpe A & van der Saag, J Mol Med 74 (1), 13 (1996); Nasu et al., Gynecol Oncol 65 (2), 304 (1997); Terol et al., Ann Oncol 14 (3), 467 (2003)), which may reach adhesion-blocking concentrations (Rose-John & Heinrich, Biochem J 300 (Pt 2), 281 (1994)). Thus, sICAM-1 produced by tumours (or stromal cells) may act to prevent circulating cytotoxic lymphocytes from transmigrating into tumour tissue and exerting anti-tumour effects promoting tumour escape from immune recognition (Gearing & Newman, Immunol Today 14 (10), 506 (1993); Sanchez-Rovira, Eur J Cancer 34 (3), 394 (1998). sICAM-1 may also stimulate tumour growth and promote angiogenesis (Gho et al. Cancer Res 59 (20), 5128 (1999); Gho et al., Cancer Res 61 (10), 4253 (2001).

Accordingly, in addition to its utility in the treatment of multiple myeloma, the antibody and/or antigen-binding fragment and/or variant, fusion or derivative as defined herein can be used to treat cancers other than multiple myeloma (such as those discussed below and particularly cancers promoted, induced or maintained by the presence of ICAM-1) when administered according to the uses and methods of the invention.

In particular, when administered in accordance with the uses and methods of the invention, the antibody and/or antigen-binding fragment and/or variant, fusion or derivative defined herein is capable of inducing apoptosis of, and/or directing antibody-dependent cell-mediated cytotoxicity (ADCC) against, cancer and/or tumour cells (such as CD20-positive and CD20-negative multiple myeloma cancer cells and tumours). In addition, when administered in accordance with the uses and methods of the invention, the antibody and/or antigen-binding fragment and/or variant, fusion or derivative as defined herein is also capable of binding soluble intercellular adhesion molecule 1 (sICAM-1), thereby inhibiting angiogenesis, cell-adhesion mediated drug-resistance and tumour-cell-escape from immunosurveillance.

The accompanying Examples demonstrate that an exemplary antibody BI-AB has significant in vivo and in vitro anti-tumour activity. In particular, BI-AB has significant activity when administered in a dose of approximately between 0.02 mg/kg and 20 mg/kg and/or in a single dosage at a frequency of once per week or less. In addition to its significant direct anti-myeloma activity, BI-AB may also act to inhibit angiogenesis-driven tumour growth and counteract tumour escape from immunosurveillance when administered in such doses and regimens.

As demonstrated in the accompanying Examples, the anti-myeloma activity of BI-AB involves both Fc-dependent and Fc-independent mechanisms of action.

In one embodiment, said antibody or antigen binding fragment is derived from an n-CoDeR® antibody library. That library is further described in WO 98/32845 (to BioInvent International AB) and is incorporated herein by reference.

Inter-Cellular Adhesion Molecule 1 (“ICAM-1”; also referred to as CD54) is an 80-114 kDa glycosylated cell-surface transmembrane protein consisting of five immunoglobulin domains, a transmembrane domain and a short cytoplasmic domain.

ICAM-1 functions as a ligand for leukocyte function associated antigen-1 (CD11a/C418), and additionally binds Mac-1 (CD11b/CD18), CD43, MUC-1, rhinovirus and fibrinogen. The predominant function of ICAM-1 is the recruitment of leucocytes to inflammatory sites, but ICAM-1 also participates in other cell-cell adhesions and in cell-activation, cell-signalling, cell-migration and cell-invasion (Rosette et al. Carcinogenesis 26, 943-950 (2005); Gho et al., Cancer Res 59, 5128-5132 (1999); Gho et al., Cancer Res 61, 4253-4257 (2001); Chirathaworn et al. J Immunol 168, 5530-5537 (2002); Berg et al., J Immunol 155, 1694-1702 (1995); Martz, Hum Immunol 18, 3-37 (1987); Poudrier & Owens, J. Exp. Med. 179, 1417-1427 (1994); Sun et al. J Cancer Res Clin Oncol 125, 28-34 (1999); Springer, Cell 76, 301-314 (1994); Siu et al., J Immunol 143, 3813-3820 (1989); Dang et al., J Immunol 144, 4082-4091 (1990); Damle et al. J Immunol 151, 2368-2379 (1993); Lane et al., J Immunol 147, 4103-4108 (1991); Ybarrondo et al., J Exp Med 179, 359-363 (1994)).

The importance of ICAM-1 in these processes is complex and has been the subject of numerous investigations. Most frequently ICAM-1 deficiency, whether conferred by genetic ablation, siRNA administration or function-blocking antibodies, has been shown to interfere with leukocyte extravasation and recruitment to inflammatory sites by decreasing or delaying cell migration and activation to different extents (Reilly et al. J Immunol 155, 529-532 (1995); Kim et al. J Immunother (1997) 30, 727-739 (2007); Smallshaw et al., J Immunother 27, 419-424 (2004); Coleman et al., J Immunother 29, 489-498 (2006); Kawano et al. Br J Haematol 79, 583-588 (1991)).

ICAM-1 exists on the cell surface as a dimer, but can also multimerize in the form of W-shapes, rings or long chains (Springer, Cell 76, 301-314 (1994); Siu, et al., J Immunol 143, 3813-3820 (1989).

Thus, by “ICAM-1” we include the monomeric form of the molecule, and dimers and multimers of the ICAM-1 monomer, including multimers in the form of W-shapes, rings or long chains.

It will be appreciated by persons skilled in the art that ICAM-1 may be derived from a human or non-human animal. In one embodiment, ICAM-1 is human.

By “binding specificity for ICAM-1” we mean an antibody or antigen-binding fragment, or variant, fusion or derivative thereof, which is capable of binding to ICAM-1 selectively. By “capable of binding selectively” we include such antibody-derived binding moieties which bind at least 10-fold more strongly to ICAM-1 than to another proteins; for example at least 50-fold more strongly or at least 100-fold more strongly. The binding moiety may be capable of binding selectively to ICAM-1 under physiological conditions, e.g. in vivo. Suitable methods for measuring relative binding strengths include immunoassays, for example where the binding moiety is an antibody (see Harlow & Lane, “Antibodies: A Laboratory”, Cold Spring Habor Laboratory Press, New York, which is incorporated herein by reference). Alternatively, binding may be assessed using competitive assays or using Biacore® analysis (Biacore International AB, Sweden).

In a further embodiment, the antibody or antigen-binding fragment, or variant, fusion or derivative thereof, binds exclusively to ICAM-1.

It will be appreciated by persons skilled in the art that the binding specificity of an antibody or antigen binding fragment thereof is conferred by the presence of complementarity determining regions (CDRs) within the variable regions of the constituent heavy and light chains. In the antibodies and antigen-binding fragments and derivatives thereof defined herein, binding specificity for ICAM-1 is conferred by the presence of one or more of the six CDRs identified as SEQ ID NOS: 1 to 6 above.

In a preferred embodiment, the antibody or antigen-binding fragment, or variant, fusion or derivative thereof of the antibody defined herein retains the binding specificity for ICAM-1 of the original antibody. By “retains the binding specificity” we mean that the antibody or antigen-binding fragment, or variant, fusion or derivative thereof as defined herein, is capable of competing for binding to ICAM-1 with the exemplary antibody of the invention (designated BI-AB; see accompanying Examples). For example, the antibody or antigen-binding fragment, or variant, fusion or derivative thereof, may bind to the same epitope on ICAM-1 as an antibody comprising the CDRs identified as SEQ ID NOS: 1 to 6.

By “epitope” it is herein intended to mean a site of a molecule to which an antibody binds, i.e. a molecular region of an antigen. An epitope may be a linear epitope, which is determined by e.g. the amino acid sequence, i.e. the primary structure, or a three-dimensional epitope, defined by the secondary structure, e.g. folding of a peptide chain into beta sheet or alpha helical, or by the tertiary structure, e.g. way which helices or sheets are folded or arranged to give a three-dimensional structure, of an antigen.

The antibody or antigen-binding fragment, or variant, fusion or derivative thereof, with binding specificity for ICAM-1 may also retain one or more of the same biological properties as the original antibody (such as the exemplary antibody, BI-AB).

Methods for determining whether a test antibody is capable of competing for binding with second antibody are well known in the art (such as, for example sandwich-ELISA or reverse-sandwich-ELISA techniques) and described, for example, in Antibodies: A Laboratory Manual, Harlow & Lane (1988, CSHL, NY, ISBN 0-87969-314-2), which is incorporated herein by reference.

In one embodiment of the uses and methods of the invention, the ICAM-1 to which the antibody or antigen-binding fragment, or a variant, fusion or derivative thereof, of the invention, is localised on the surface of a cell. Such binding specificity may be determined by methods well known in the art, such as enzyme-linked immunosorbent assay (ELISA), immunohistochemistry, immunoprecipitation, Western blot and flow cytometry using transfected cells expressing ICAM-1. Methods suitable for determining binding specificity are described in the accompanying Examples.

ICAM-1 is expressed constitutively at low levels on vascular endothelium, epithelial cells, fibroblasts, keratinocytes, leukocytes as well as conventional antigen presenting cells (APC) (reviewed in Roebuck. & Finnegan, J Leukoc Biol, 66, 876-888 (1999)). Cell surface ICAM-1 expression can be induced by several cytokines and pro-inflammatory agents, such as IFN-γ, TNF-α, lipopolysaccharide (LPS), oxygen radicals or hypoxia (Roebuck & Finnegan, J Leukoc Biol 66, 876-888 (1999)). ICAM-1 may additionally be shed from cells by proteolysis or by alternative splicing depending on cell type (Dang et al., J Immunol 144, 4082-4091 (1990); Damle et al. J Immunol 151, 2368-2379 (1993); Lane et al., J Immunol 147, 4103-4108 (1991); Ybarrondo, et al., J Exp Med 179, 359-363 (1994)).

Conveniently, the ICAM-1 is localised on the surface of a cancer cell; typically a cancer cell selected from the group consisting or comprising of: a myeloma cell; a melanoma cell; a lung cancer cell; a gastric cancer cell; a bladder cancer cell; a breast cancer cell; a prostate cancer cell; a lymphoma cell; a renal cancer cell; a hepatocellular carcinoma cell. In a preferred embodiment, the cancer cell is a myeloma cell.

In an alternative embodiment, the ICAM-1 is soluble—that is, the ICAM-1 is not localised on the surface of a cell and is present in bodily fluids in the circulation. As discussed above, in addition to increased cell-surface expression of ICAM-1, cancer patients have elevated soluble ICAM-1 (sICAM-1) in the circulation which may reach adhesion-blocking concentrations which may act to prevent circulating cytotoxic lymphocytes from transmigrating into tumour tissue and exerting anti-tumour effects promoting tumour escape from immune recognition, and also stimulate tumour growth and promote angiogenesis. Thus, in an embodiment, the present uses and methods can be used to prevent and/or reduce one or more of the activities of soluble ICAM-1, thereby preventing and/or reducing tumour escape, tumour growth and/or angiogenesis.

Accordingly, as the antibody defined herein can modulate immune recognition and the immune response, in an alternative embodiment the uses and methods of the invention can be used to treat inflammatory diseases or conditions. In a preferred embodiment, the inflammatory diseases or conditions are selected from the group consisting or comprising of atherosclerosis; asthma; transplant rejection; psoriases.

It is now known that multiple myeloma patients that have developed resistance to chemotherapy have increased (myeloma cell) expression of ICAM-1 (Schmidmaier et al., Int J Biol Markers 21 (4), 218 (2006)), and that ICAM-1-mediated adhesion of myeloma cells to bone marrow stroma can increase survival of myeloma cells and induce primary multidrug resistance (Schmidmaier et al., Int J Biol Markers 21 (4), 218 (2006)).

In an even more preferred embodiment of the methods and uses of the invention, the antibody and/or antigen-binding fragment and/or variant, fusion or derivative as defined herein is used in methods, uses and/or compositions for the treatment of patients with relapsed refractory multiple myeloma, and those who have multiple myeloma and have developed resistance to chemotherapy and related treatments.

By “antibody” we include substantially intact antibody molecules, as well as chimeric antibodies, humanised antibodies, human antibodies (wherein at least one amino acid is mutated relative to the naturally occurring human antibodies), single chain antibodies, bi-specific antibodies, antibody heavy chains, antibody light chains, homo-dimers and heterodimers of antibody heavy and/or light chains, and antigen binding fragments and derivatives of the same.

The term ‘antibody’ also includes all classes of antibodies, including IgG, IgA, IgM, IgD and IgE. Thus, the antibody may be an IgG molecule, such as an IgG1, IgG2, IgG3, or IgG4 molecule. Preferably, the antibody of the invention is an IgG molecule, or an antigen-binding fragment, or variant, fusion or derivative thereof.

In one embodiment of the uses and methods of the invention, the antibody comprises or consists of an intact antibody. Alternatively, the antibody or antigen-binding fragment, or variant, fusion or derivative thereof, may consist essentially of an intact antibody. By “consist essentially of” we mean that the antibody or antigen-binding fragment, variant, fusion or derivative thereof consists of a portion of an intact antibody sufficient to display binding specificity for ICAM-1.

In one embodiment of the uses and methods of the invention, the antibody is a non-naturally occurring antibody. Of course, where the antibody is a naturally occurring antibody, it is provided in an isolated form (i.e. distinct from that in which it is found in nature).

The variable heavy (V_{H}) and variable light (V_{L}) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent-parented antibody (Morrison at al (1984) Proc. Natl. Acad. Sci. USA 81, 6851-6855).

Antigenic specificity is conferred by variable domains and is independent of the constant domains, as known from experiments involving the bacterial expression of antibody fragments, all containing one or more variable domains. These molecules include Fab-like molecules (Better et al (1988) Science 240, 1041); Fv molecules (Skerra et al (1988) Science 240, 1038); single-chain Fv (ScFv) molecules where the V_{H} and V_{L} partner domains are linked via a flexible oligopeptide (Bird et al (1988) Science 242, 423; Huston et al (1988) Proc. Natl. Acad. Sci. USA 85, 5879) and single domain antibodies (dAbs) comprising isolated V domains (Ward et al (1989) Nature 341, 544). A general review of the techniques involved in the synthesis of antibody fragments which retain their specific binding sites is to be found in Winter & Milstein (1991) Nature 349, 293-299.

Thus, by “antigen-binding fragment” we mean a functional fragment of an antibody that is capable of binding to ICAM-1.

Exemplary antigen-binding fragments may be selected from the group consisting of Fv fragments (e.g. single chain Fv and disulphide-bonded Fv), and Fab-like fragments (e.g. Fab fragments, Fab′ fragments and F(ab)₂ fragments).

In one embodiment of the uses and methods of the invention, the antigen-binding fragment is an scFv.

The advantages of using antibody fragments, rather than whole antibodies, are several-fold.

The smaller size of the fragments may lead to improved pharmacological properties, such as better penetration of solid tissue. Moreover, antigen-binding fragments such as Fab, Fv, ScFv and dAb antibody fragments can be expressed in and secreted from E. coli, thus allowing the facile production of large amounts of the said fragments.

Also included within the scope of the invention are modified versions of antibodies and an antigen-binding fragments thereof, e.g. modified by the covalent attachment of polyethylene glycol or other suitable polymer.

Methods of generating antibodies and antibody fragments are well known in the art. For example, antibodies may be generated via any one of several methods which employ induction of in vivo production of antibody molecules, screening of immunoglobulin libraries (Orlandi. et al, 1989. Proc. Natl. Acad. Sci. U.S.A. 86:3833-3837; Winter et al., 1991, Nature 349:293-299) or generation of monoclonal antibody molecules by cell lines in culture. These include, but are not limited to, the hybridoma technique, the human B-cell hybridoma technique, and the Epstein-Barr virus (EBV)-hybridoma technique (Kohler et al., 1975. Nature 256:4950497; Kozbor et al., 1985. J. Immunol. Methods 81:31-42; Cote et al., 1983. Proc. Natl. Acad. Sci. USA 80:2026-2030; Cole et al., 1984. Mol. Cell. Biol. 62:109-120).

Conveniently, the invention provides an antibody or antigen-binding fragment, or a variant, fusion or derivative thereof, wherein the antibody is a recombinant antibody (i.e. wherein it is produced by recombinant means).

In a preferred embodiment of the uses and methods of the invention, the antibody is a monoclonal antibody.

Suitable monoclonal antibodies to selected antigens may be prepared by known techniques, for example those disclosed in “Monoclonal Antibodies: A manual of techniques”, H Zola (CRC Press, 1988) and in “Monoclonal Hybridoma Antibodies: Techniques and Applications”, J G R Hurrell (CRC Press, 1982), which are incorporated herein by reference.

Antibody fragments can also be obtained using methods well known in the art (see, for example, Harlow & Lane, 1988, “Antibodies: A Laboratory Manual”, Cold Spring Harbor Laboratory, New York, which is incorporated herein by reference). For example, antibody fragments for use in the methods and uses of the present invention can be prepared by proteolytic hydrolysis of the antibody or by expression in E. coli or mammalian cells (e.g. Chinese hamster ovary cell culture or other protein expression systems) of DNA encoding the fragment. Alternatively, antibody fragments can be obtained by pepsin or papain digestion of whole antibodies by conventional methods.

Preferably, the invention provides a use or method wherein the antibody or antigen-binding fragment thereof is a human antibody or humanised antibody.

It will be appreciated by persons skilled in the art that for human therapy or diagnostics, humanised antibodies may be used. Humanised forms of non-human (e.g. murine) antibodies are genetically engineered chimeric antibodies or antibody fragments having minimal-portions derived from non-human antibodies. Humanised antibodies include antibodies in which complementary determining regions of a human antibody (recipient antibody) are replaced by residues from a complementary determining region of a non human species (donor antibody) such as mouse, rat of rabbit having the desired functionality. In some instances, Fv framework residues of the human antibody are replaced by corresponding non-human residues. Humanised antibodies may also comprise residues which are found neither in the recipient antibody nor in the imported complementarity determining region or framework sequences. In general, the humanised antibody will comprise substantially all of at least one, and typically two, variable domains, in which all or substantially all of the complementarity determining regions correspond to those of a non-human antibody and all, or substantially all, of the framework regions correspond to those of a relevant human consensus sequence. Humanised antibodies optimally also include at least a portion of an antibody constant region, such as an Fc region, typically derived from a human antibody (see, for example, Jones et al., 1986. Nature 321:522-525; Riechmann et al., 1988, Nature 332:323-329; Presta, 1992, Curr. Op. Struct. Biol. 2:593-596, which are incorporated herein by reference).

Methods for humanising non-human antibodies are well known in the art. Generally, the humanised antibody has one or more amino acid residues introduced into it from a source which is non-human. These non-human amino acid residues, often referred to as imported residues, are typically taken from an imported variable domain. Humanisation can be essentially performed as described (see, for example, Jones et al., 1986, Nature 321:522-525; Reichmann et al., 1988. Nature 332:323-327; Verhoeyen et al., 1988, Science 239:1534-15361; U.S. Pat. No. 4,816,567, which are incorporated herein by reference) by substituting human complementarity determining regions with corresponding rodent complementarity determining regions. Accordingly, such humanised antibodies are chimeric antibodies, wherein substantially less than an intact human variable domain has been substituted by the corresponding sequence from a non-human species. In practice, humanised antibodies may be typically human antibodies in which some complementarity determining region residues and possibly some framework residues are substituted by residues from analogous sites in rodent antibodies.

Human antibodies can also be identified using various techniques known in the art, including phage display libraries (see, for example, Hoogenboom & Winter, 1991, J. Mol. Biol. 227:381; Marks et al., 1991, J. Mol. Biol. 222:581; Cole at al., 1985, In: Monoclonal antibodies and Cancer Therapy, Alan R. Liss, pp. 77; Boerner et al., 1991. J. Immunol. 147:86-95, Soderlind et al., 2000, Nat Biotechnol 18:852-6 and WO 98/32845 which are incorporated herein by reference).

Once suitable antibodies are obtained, they may be tested for activity, such as binding specificity or a biological activity of the antibody, for example by ELISA, immunohistochemistry, flow cytometry, immunoprecipitation, Western blots, etc. The biological activity may be tested in different assays with readouts for that particular feature.

Examples of one or more biological activity of the antibody for use in the method and uses of the invention are:
a) reducing and/or preventing tumour cell proliferation;
b) inducing tumour cell apoptosis;
c) inducing and/or increasing antibody-dependent cell cytotoxicity towards one or more tumour cell.

Examples of suitable assays for testing said biological activity (in vitro or in vivo) is known in the art and also given in the accompanying Examples.

In one embodiment of the uses and methods of the invention, the antibody, fragment, variant, fusion or derivative comprises, consists essentially of, or consists of the amino acid sequences of SEQ ID NOS: 1 to 6.

In a particularly preferred embodiment of the uses and methods of the invention, the antibody or antigen-binding fragment, or variant, fusion or derivative thereof, comprises a heavy chain variable region comprising, consisting essentially of, or consisting of the following CDRs:

[SEQ ID NO: 1]
FSNAWMSWVRQAPG;
and
[SEQ ID NO: 2]
AFIWYDGSNKYYADSVKGR;
and
[SEQ ID NO: 3]
ARYSGWYFDY.

Preferably, the heavy chain variable region comprises, consists essentially of, or consists of the amino acid sequence of SEQ ID NO: 7.

In one embodiment of the methods and uses of the invention, the invention provides an antibody or antigen-binding fragment comprising a light chain variable region comprising, consisting essentially of, or consisting of the following CDRs:

[SEQ ID NO: 4]
CTGSSSNIGAGYDVH;
and/or
[SEQ ID NO: 5]
DNNNRPS;
and/or
[SEQ ID NO: 6]
CQSYDSSLSAWL.

Preferably, the light chain variable region comprises, consists essentially of, or consists of the amino acid sequence of SEQ ID NO: 8.

In a particularly preferred embodiment of the uses and methods of the invention, the antibody or antigen-binding fragment, or variant, fusion or derivative thereof, comprises a heavy chain variable region as defined herein and a light chain variable region as defined herein.

For example, it is preferred that the antibody or antigen-binding fragment, or the variant, fusion or derivative thereof, comprises a heavy chain variable region comprising the amino acid sequence of SEQ ID NO: 7 and a light chain variable region comprising the amino acid sequence of SEQ ID NO: 8.

The term ‘amino acid’ as used herein includes the standard twenty genetically-encoded amino acids and their corresponding stereoisomers in the ‘D’ form (as compared to the natural ‘L’ form), omega-amino acids other naturally-occurring amino acids, unconventional amino acids (e.g. α,α-disubstituted amino acids, N-alkyl amino acids, etc.) and chemically derivatised amino acids (see below).

When an amino acid is being specifically enumerated, such as ‘alanine’ or ‘Ala’ or ‘A’, the term refers to both L-alanine and D-alanine unless explicitly stated otherwise. Other unconventional amino acids may also be suitable components for polypeptides of the present invention, as long as the desired functional property is retained by the polypeptide. For the peptides shown, each encoded amino acid residue, where appropriate, is represented by a single letter designation, corresponding to the trivial name of the conventional amino acid.

In one embodiment, the polypeptides as defined herein comprise or consist of L-amino acids.

It will be appreciated by persons skilled in the art that the methods and uses of the invention encompass variants, fusions and derivatives of the defined polypeptides, as well as fusions of a said variants or derivatives, provided such variants, fusions and derivatives have binding specificity for ICAM-1.

Variants may be made using the methods of protein engineering and site-directed mutagenesis well known in the art using the recombinant polynucleotides (see example, see Molecular Cloning: a Laboratory Manual, 3rd edition, Sambrook & Russell, 2001, Cold Spring Harbor Laboratory Press, which is incorporated herein by reference).

By ‘fusion’ of said polypeptide we include a polypeptide fused to any other polypeptide. For example, the said polypeptide may be fused to a polypeptide such as glutathione-S-transferase (GST) or protein A in order to facilitate purification of said polypeptide. Examples of such fusions are well known to those skilled in the art. Similarly, the said polypeptide may be fused to an oligo-histidine tag such as His6 or to an epitope recognised by an antibody such as the well-known Myc-tag epitope. Fusions to any variant or derivative of said polypeptide are also included in the scope of the invention. It will be appreciated that fusions (or variants or derivatives thereof) which retain desirable properties, such as have binding specificity for ICAM-1, are preferred.

The fusion may comprise a further portion which confers a desirable feature on the said polypeptide of the invention; for example, the portion may be useful in detecting or isolating the polypeptide, or promoting cellular uptake of the polypeptide. The portion may be, for example, a biotin moiety, a radioactive moiety, a fluorescent moiety, for example a small fluorophore or a green fluorescent protein (GFP) fluorophore, as well known to those skilled in the art. The moiety may be an immunogenic tag, for example a Myc-tag, as known to those skilled in the art or may be a lipophilic molecule or polypeptide domain that is capable of promoting cellular uptake of the polypeptide, as known to those skilled in the art.

By ‘variants’ of the polypeptide we include insertions, deletions and substitutions, either conservative or non-conservative. In particular we include variants of the polypeptide where such changes do not substantially alter the activity of the said polypeptide. In particular, we include variants of the polypeptide where such changes do not substantially alter the binding specificity for ICAM-1.

The polypeptide variant may have an amino acid sequence which has at least 75% identity with one or more of the amino acid sequences given above, for example at least 80%, at least 90%, at least 95%, at least 96%, at least 97%, at least 98%, or at least 99% identity with one or more of the amino acid sequences specified above.

The percent sequence identity between two polypeptides may be determined using suitable computer programs, for example the GAP program of the University of Wisconsin Genetic Computing Group and it will be appreciated that percent identity is calculated in relation to polypeptides whose sequences have been aligned optimally.

The alignment may alternatively be carried out using the Clustal W program (as described in Thompson et al., 1994, Nuc. Acid Res. 22:4673-4680, which is incorporated herein by reference).

The parameters used may be as follows:
Fast pairwise alignment parameters: K-tuple(word) size; 1, window size; 5, gap penalty; 3, number of top diagonals; 5. Scoring method: x percent.
Multiple alignment parameters: gap open penalty; 10, gap extension penalty; 0.05.
Scoring matrix: BLOSUM.

Alternatively, the BESTFIT program may be used to determine local sequence alignments.

The polypeptide, variant, fusion or derivative used in the methods or uses of the invention may comprise one or more amino acids which have been modified or derivatised.

Chemical derivatives of one or more amino acids may be achieved by reaction with a functional side group. Such derivatised molecules include, for example, those molecules in which free amino groups have been derivatised to form amine hydrochlorides, p-toluene sulphonyl groups, carboxybenzoxy groups, t-butyloxycarbonyl groups, chloroacetyl groups or formyl groups. Free carboxyl groups may be derivatised to form salts, methyl and ethyl esters or other types of esters and hydrazides. Free hydroxyl groups may be derivatised to form O-acyl or O-alkyl derivatives. Also included as chemical derivatives are those peptides which contain naturally occurring amino acid derivatives of the twenty standard amino acids. For example: 4-hydroxyproline may be substituted for proline; 5-hydroxylysine may be substituted for lysine; 3-methylhistidine may be substituted for histidine; homoserine may be substituted for serine and ornithine for lysine. Derivatives also include peptides containing one or more additions or deletions as long as the requisite activity is maintained. Other included modifications are amidation, amino terminal acylation (e.g. acetylation or thioglycolic acid amidation), terminal carboxylamidation (e.g. with ammonia or methylamine), and the like terminal modifications.

It will be further appreciated by persons skilled in the art that peptidomimetic compounds may also be useful. Thus, by ‘polypeptide’ we include peptidomimetic compounds which are capable of binding ICAM-1. The term ‘peptidomimetic’ refers to a compound that mimics the conformation and desirable features of a particular peptide as a therapeutic agent.

For example, the polypeptides of the invention include not only molecules in which amino acid residues are joined by peptide (—CO—NH—) linkages but also molecules in which the peptide bond is reversed. Such retro-inverso peptidomimetics may be made using methods known in the art, for example such as those described in Meziere et al. (1997) J. Immunol. 159, 3230-3237, which is incorporated herein by reference. This approach involves making pseudopeptides containing changes involving the backbone, and not the orientation of side chains. Retro-inverse peptides, which contain NH—CO bonds instead of CO—NH peptide bonds, are much more resistant to proteolysis. Alternatively, the polypeptide of the invention may be a peptidomimetic compound wherein one or more of the amino acid residues are linked by a -y(CH₂NH)— bond in place of the conventional amide linkage.

In a further alternative, the peptide bond may be dispensed with altogether provided that an appropriate linker moiety which retains the spacing between the carbon atoms of the amino acid residues is used; it may be advantageous for the linker moiety to have substantially the same charge distribution and substantially the same planarity as a peptide bond.

It will be appreciated that the polypeptide may conveniently be blocked at its N- or C-terminus so as to help reduce susceptibility to exoproteolytic digestion.

A variety of uncoded or modified amino acids such as D-amino acids and N-methyl amino acids have also been used to modify mammalian peptides. In addition, a presumed bioactive conformation may be stabilised by a covalent modification, such as cyclisation or by incorporation of lactam or other types of bridges, for example see Veber et al., 1978, Proc. Natl. Acad. Sci. USA 75:2636 and Thursell et al., 1983, Biochem. Biophys. Res. Comm. 111:166, which are incorporated herein by reference.

A common theme among many of the synthetic strategies has been the introduction of some cyclic moiety into a peptide-based framework. The cyclic moiety restricts the conformational space of the peptide structure and this frequently results in an increased specificity of the peptide for a particular biological receptor. An added advantage of this strategy is that the introduction of a cyclic moiety into a peptide may also result in the peptide having a diminished sensitivity to cellular peptidases.

Thus, exemplary polypeptides useful in the methods and uses of the invention comprise terminal cysteine amino acids. Such a polypeptide may exist in a heterodetic cyclic form by disulphide bond formation of the mercaptide groups in the terminal cysteine amino acids or in a homodetic form by amide peptide bond formation between the terminal amino acids. As indicated above, cyclising small peptides through disulphide or amide bonds between the N- and C-terminus cysteines may circumvent problems of specificity and half-life sometime observed with linear peptides, by decreasing proteolysis and also increasing the rigidity of the structure, which may yield higher specificity compounds. Polypeptides cyclised by disulphide bonds have free amino and carboxy-termini which still may be susceptible to proteolytic degradation, while peptides cyclised by formation of an amide bond between the N-terminal amine and C-terminal carboxyl and hence no longer contain free amino or carboxy termini. Thus, the peptides of the present invention can be linked either by a C-N linkage or a disulphide linkage.

The present invention is not limited in any way by the method of cyclisation of peptides, but encompasses peptides whose cyclic structure may be achieved by any suitable method of synthesis. Thus, heterodetic linkages may include, but are not limited to formation via disulphide, alkylene or sulphide bridges. Methods of synthesis of cyclic homodetic peptides and cyclic heterodetic peptides, including disulphide, sulphide and alkylene bridges, are disclosed in U.S. Pat. No. 5,643,872, which is incorporated herein by reference. Other examples of cyclisation methods are discussed and disclosed in U.S. Pat. No. 6,008,058, which is incorporated herein by reference.

A further approach to the synthesis of cyclic stabilised peptidomimetic compounds is ring-closing metathesis (RCM). This method involves steps of synthesising a peptide precursor and contacting it with an RCM catalyst to yield a conformationally restricted peptide. Suitable peptide precursors may contain two or more unsaturated C—C bonds. The method may be carried out using solid-phase-peptide-synthesis techniques. In this embodiment, the precursor, which is anchored to a solid support, is contacted with a RCM catalyst and the product is then cleaved from the solid support to yield a conformationally restricted peptide.

Another approach, disclosed by D. H. Rich in Protease Inhibitors, Barrett and Selveson, eds., Elsevier (1986), which is incorporated herein by reference, has been to design peptide mimics through the application of the transition state analogue concept in enzyme inhibitor design. For example, it is known that the secondary alcohol of staline mimics the tetrahedral transition state of the scissile amide bond of the pepsin substrate.

In summary, terminal modifications are useful, as is well known, to reduce susceptibility by proteinase digestion and therefore to prolong the half-life of the peptides in solutions, particularly in biological fluids where proteases may be present. Polypeptide cyclisation is also a useful modification because of the stable structures formed by cyclisation and in view of the biological activities observed for cyclic peptides.

Thus, in one embodiment the polypeptide used in the methods and uses of the invention is cyclic. However, in a alternative embodiment, the polypeptide is linear.

In a preferred embodiment of the methods and uses of the invention, the antibody or antigen-binding fragment, or variant, fusion or derivative thereof, is capable of specifically binding ICAM-1 localised on the surface of a cell and inhibiting and/or preventing proliferation of that cell.

In an alternative embodiment, the antibody or antigen-binding fragment, or a variant, fusion or derivative thereof, is capable of specifically binding ICAM-1 localised on the surface of a cell and inducing apoptosis of that cell.

In an alternative embodiment, the antibody or antigen-binding fragment, or variant, fusion or derivative thereof, is capable of specifically binding ICAM-1 localised on the surface of a cell and inducing antibody-dependent cell cytotoxicity against that cell.

Conveniently, the antibody or antigen-binding fragment, or the variant, fusion or derivative thereof has efficacy in the treatment of cancer, typically wherein the cancer is selected from the group consisting of multiple myeloma; melanoma; lung cancer; gastric cancer; bladder cancer; breast cancer; prostate cancer; lymphoma; renal cancer; hepatocellular carcinoma.

Such efficacy may be determined in suitable animal models, such as arthritis models in mice (see Examples).

In a particularly preferred embodiment, the cancer is multiple myeloma.

The antibody, antigen-binding fragment, and/or fusion, derivative or variants thereof, and medicaments of the invention may be delivered using an injectable sustained-release drug delivery system. These are designed specifically to reduce the frequency of injections. An example of such a system is Nutropin Depot which encapsulates recombinant human growth hormone (rhGH) in biodegradable microspheres that, once injected, release rhGH slowly over a sustained period. Preferably, delivery is performed intra-muscularly (i.m.) and/or sub-cutaneously (s.c.) and/or intravenously (i.v.).

The antibody, antigen-binding fragment, and/or fusion, derivative or variants thereof, and medicaments of the invention can be administered by a surgically implanted device that releases the drug directly to the required site. For example, Vitrasert releases ganciclovir directly into the eye to treat CMV retinitis. The direct application of this toxic agent to the site of disease achieves effective therapy without the drug's significant systemic side-effects.

Electroporation therapy (EPT) systems can also be employed for the administration of the antibody, antigen-binding fragment, and/or fusion, derivative or variants thereof, medicaments and pharmaceutical compositions of the invention. A device which delivers a pulsed electric field to cells increases the permeability of the cell membranes to the drug, resulting in a significant enhancement of intracellular drug delivery.

The antibody, antigen-binding fragment, and/or fusion, derivative or variants thereof, and medicaments of the invention can also be delivered by electro-incorporation (EI). EI occurs when small particles of up to 30 microns in diameter on the surface of the skin experience electrical pulses identical or similar to those used in electroporation. In EI, these particles are driven through the stratum corneum and into deeper layers of the skin. The particles can be loaded or coated with drugs or genes or can simply act as “bullets” that generate pores in the skin through which the drugs can enter.

An alternative method of delivery of the antibody, antigen-binding fragment, and/or fusion, derivative or variants thereof, and medicaments of the invention is the ReGel® injectable system that is thermo-sensitive. Below body temperature, ReGel is an injectable liquid while at body temperature it immediately forms a gel reservoir that slowly erodes and dissolves into known, safe, biodegradable polymers. The active substance is delivered over time as the biopolymers dissolve.

The antibody, antigen-binding fragment, and/or fusion, derivative or variants thereof, and medicaments of the invention can also be delivered orally. The process employs a natural process for oral uptake of vitamin B₁₂ and/or vitamin D in the body to co-deliver proteins and peptides. By riding the vitamin B₁₂ and/or vitamin D uptake system, the antibody, antigen-binding fragment, and/or fusion, derivative or variants thereof, and medicaments of the invention can move through the intestinal wall. Complexes are synthesised between vitamin B₁₂ analogues and/or vitamin D analogues and the drug that retain both significant affinity for intrinsic factor (IF) in the vitamin B₁₂ portion/vitamin D portion of the complex and significant bioactivity of the active substance of the complex.

The antibody, antigen-binding fragment, and/or fusion, derivative or variants thereof, and medicaments of the invention can be introduced to cells by “Trojan peptides”. These are a class of polypeptides called penetratins which have translocating properties and are capable of carrying hydrophilic compounds across the plasma membrane. This system allows direct targeting of oligopeptides to the cytoplasm and nucleus, and may be non-cell type specific and highly efficient. See Derossi et al. (1998), Trends Cell Biol 8, 84-87.

Preferably, the medicament of the present invention is a unit dosage containing a daily dose or unit, daily sub-dose or an appropriate fraction thereof, of the active ingredient.

The antibody, antigen-binding fragment, and/or fusion, derivative or variants thereof and/or medicaments of the invention will normally be administered orally or by any parenteral route, in the form of a pharmaceutical composition comprising the active ingredient, optionally in the form of a non-toxic organic, or inorganic, acid, or base, addition salt, in a pharmaceutically acceptable dosage form. Depending upon the disorder and patient to be treated, as well as the route of administration, the compositions may be administered at varying doses.

In human therapy, the antibody, antigen-binding fragment, and/or fusion, derivative or variants thereof, and medicaments of the invention can be administered alone but will generally be administered in admixture with a suitable pharmaceutical excipient, diluent or carrier selected with regard to the intended route of administration and standard pharmaceutical practice.

For example, the antibody, antigen-binding fragment, and/or fusion, derivative or variants thereof, and medicaments of the invention can be administered orally, buccally or sublingually in the form of tablets, capsules, ovules, elixirs, solutions or suspensions, which may contain flavouring or colouring agents, for immediate-, delayed- or controlled-release applications. The antibody, antigen-binding fragment, and/or fusion, derivative or variants thereof, and medicaments of the invention may also be administered via intracavernosal injection.

Such tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, cellulose, milk sugar or high molecular weight polyethylene glycols. For aqueous suspensions and/or elixirs, the antibody, antigen-binding fragment, and/or fusion, derivative or variants thereof, medicaments and pharmaceutical compositions of the invention may be combined with various sweetening or flavouring agents, colouring matter or dyes, with emulsifying and/or suspending agents and with diluents such as water, ethanol, propylene glycol and glycerin, and combinations thereof.

The antibody, antigen-binding fragment, and/or fusion, derivative or variants thereof, and medicaments of the invention can also be administered parenterally, for example, intravenously, intra-arterially, intraperitoneally, intra-thecally, intraventricularly, intrasternally, intracranially, intra-muscularly or subcutaneously, or they may be administered by infusion techniques. They are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Medicaments and pharmaceutical compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The medicaments and pharmaceutical compositions may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

The antibody, antigen-binding fragment, and/or fusion, derivative or variants thereof, and medicaments of the invention can also be administered intranasally or by inhalation and are conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoro-ethane, a hydrofluoroalkane such as 1,1,1,2-tetrafluoroethane (HFA 134A3 or 1,1,1,2,3,3,3-heptafluoropropane (HFA 227EA3), carbon dioxide or other suitable gas. In the case of a pressurised aerosol, the dosage unit may be determined by providing a valve to deliver a metered amount. The pressurised container, pump, spray or nebuliser may contain a solution or suspension of the active agent, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of an antibody, antigen-binding fragment, and/or fusion, derivative or variants thereof, of the invention and a suitable powder base such as lactose or starch.

Aerosol or dry powder formulations are preferably arranged so that each metered dose or “puff” contains an effective amount of an agent or polynucleotide of the invention for delivery to the patient. It will be appreciated that he overall daily dose with an aerosol will vary from patient to patient, and may be administered in a single dose or, more usually, in divided doses throughout the day.

Alternatively, the antibody, antigen-binding fragment, and/or fusion, derivative or variants thereof, and medicaments of the invention can be administered in the form of a suppository or pessary, or they may be applied topically in the form of a lotion, solution, cream, gel, ointment or dusting powder. The antibody, antigen-binding fragment, and/or fusion, derivative or variants thereof, and medicaments of the invention may also be transdermally administered, for example, by the use of a skin patch. They may also be administered by the ocular route, particularly for treating diseases of the eye.

For ophthalmic use, the antibody, antigen-binding fragment, and/or fusion, derivative or variants thereof, and medicaments of the invention can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the antibody, antigen-binding fragment, and/or fusion, derivative or variants thereof, and medicaments of the invention can be formulated as a suitable ointment containing the active agent suspended or dissolved in, for example, a mixture with one or more of the following: mineral oil, liquid petrolatum, white petrolatum, propylene glycol, polyoxyethylene polyoxypropylene agent, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

Formulations suitable for topical administration in the mouth include lozenges comprising the active ingredient in a flavoured basis, usually sucrose and acacia or tragacanth; pastilles comprising the active ingredient in an inert basis such as gelatin and glycerin, or sucrose and acacia; and mouth-washes comprising the active ingredient in a suitable liquid carrier.

Generally, in humans, oral or parenteral administration of the antibody, antigen-binding fragment, and/or fusion, derivative or variants thereof, medicaments and pharmaceutical compositions of the invention is the preferred route, being the most convenient.

For veterinary use, the antibody, antigen-binding fragment, and/or fusion, derivative or variants thereof, and medicaments of the invention are administered as a suitably acceptable formulation in accordance with normal veterinary practice and the veterinary surgeon will determine the dosing regimen and route of administration which will be most appropriate for a particular animal.

The antibody or antigen-binding fragment, or variant, fusion or derivative thereof, as defined herein may be formulated as described in the accompanying Examples.

According to a further embodiment of the invention, the effectiveness of antibody, antigen binding fragment, variant, fusion or derivative thereof according to the present invention in alleviating the symptoms, preventing or treating disease may be improved by serial administering or administration in combination with another agent that is effective for the same clinical indication, such as another antibody or a fragment thereof directed against a different epitope than that of the antibody according to the invention, or one or more conventional therapeutic agents known for the intended therapeutic indication.

For example, the additional agent may be one or more agent selected from the group consisting or comprising of: Revlimid; Talibomib; Melphalan; Bortesomib; Velcade; cytostatic agents; Predison (a hormonal therapy) or similar hormonal drugs; tyrosine kinase inhibitors

As discussed above, when administered according to the methods and uses of the invention, the antibody and/or antigen-binding fragment and/or variant, fusion or derivative as defined herein is capable of inducing apoptosis of, and/or directing antibody-dependent cell-mediated cytotoxicity (ADCC) against, cancer and/or tumour cells (such as CD20-positive and CD20-negative multiple myeloma cancer cells and tumours). In addition, the antibody and/or antigen-binding fragment and/or variant, fusion or derivative is capable of binding soluble intercellular adhesion molecule 1 (sICAM-1), thereby inhibiting angiogenesis, cell-adhesion mediated drug-resistance and tumour-cell-escape from immunosurveillance.

The accompanying Examples demonstrate that an exemplary antibody as defined herein (termed antibody “BI-AB”) has significant in vivo and in vitro anti-tumour activity when administered according to the methods and uses of the invention. In addition to its significant direct anti-myeloma activity, BI-AB may also act to inhibit angiogenesis-driven tumour growth and counteract tumour escape from immunosurveillance.

In a particularly preferred embodiment, the cancer is multiple myeloma.

As used herein, the singular forms “a”, “and”, and “the” include plural referents unless the context clearly dictates otherwise. Thus, for example, reference to “an antibody” includes a plurality of such antibodies and reference to “the dosage” includes reference to one or more dosages and equivalents thereof known to those skilled in the art, and so forth.

Preferred, non-limiting examples which embody certain aspects of the invention will now be described, with reference to the following figures:

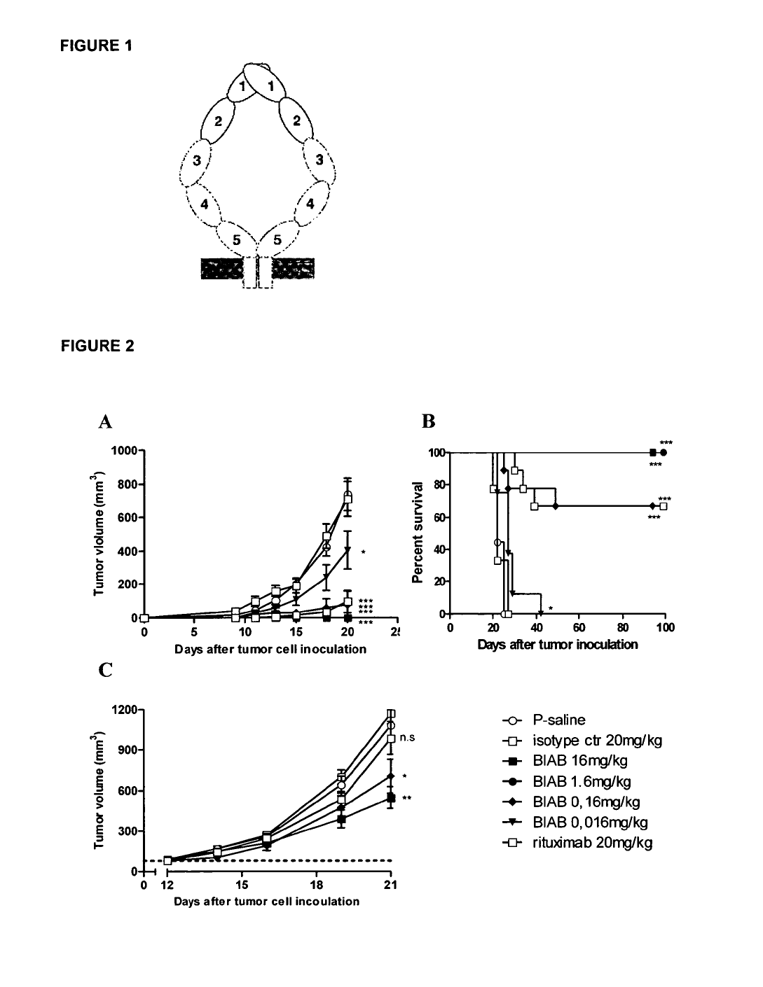

FIG. 1: Structure of ICAM-1 (intercellular adhesion molecule 1).

FIG. 2: BI-AB shows significant in vivo anti-myeloma efficacy and potency in SCID/ARH-77 myeloma xenograft models.
Tumour cells were injected subcutaneously into the left flank of SCID mice. Mice received twice-weekly intraperitoneal injections with BI-AB at doses of 16, 1.6, 0.16, 0.016 mg/kg commencing as indicated below. There were 8 to 10 animals per treatment group.
A and B: Early tumour model; treatment started 1 day after myeloma cell inoculation and continued until tumour volumes reached the ethical limit. (A) Tumour volume as a function of antibody dose. (B) Kaplan-Meier survival graph as a function of antibody dose.
(C) Established xenograft model; treatment with antibodies started when tumours reached a volume of approximately 100 mm³. Antibody treatment did not affect animal body weight and there were no adverse effects in any of the antibody-treated mice in the experiments. Statistical significance was calculated relative to control antibody treatment using Student t test (tumour volume) or the log-rank test (mouse survival) using Graphpad Prism software. Statistical significance was considered for *p<0.05, **p<0.01, and ***p<0.001. Graphs illustrate representative experiments out of several performed.

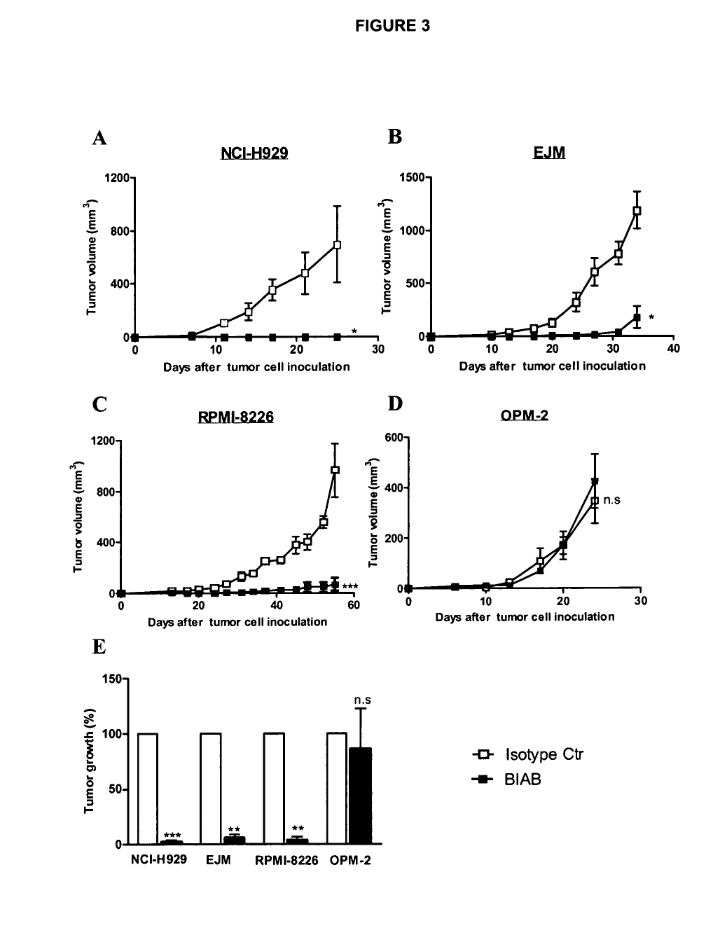

FIG. 3: BI-AB shows significant and ICAM-1-dependent anti-myeloma activity in vivo.
NCI-H929, EJM, RPMI-8226, or OPM-2 myeloma cells were injected subcutaneously into the left flank of SCID mice at Day 0. Antibody treatment with 2 mg/kg BI-AB or control IgG₁ was started at Day 1 and was continued on a twice-weekly intraperitoneal dosing regimen. Mice were sacrificed when tumour sizes reached the ethical limit. BI-AB had no effect on tumour growth in animals xenografted with the ICAM-1-negative cell line OPM-2, demonstrating that anti-myeloma activity was ICAM-1-dependent.
Graphs A-D show data from one representative experiment out of two performed. Graph E shows pooled and normalized data from two independent experiments (n=8 to 10 animals per treatment group). Statistical significance was calculated relative to control antibody treatment using Student t test. Statistical significance was considered for *p<0.05, **p <0.01, and ***p<0.001. ICAM-1-intercellular adhesion molecule 1; SCID=severe combined immunodeficiency

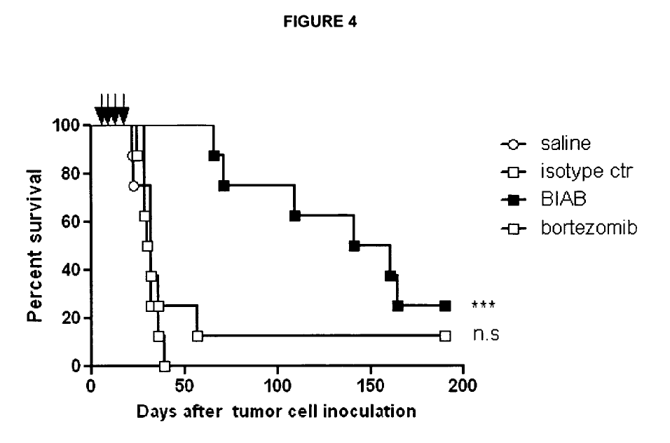

FIG. 4: BI-AB confers protection against advanced experimental multiple myeloma. SCID mice were irradiated 5 days before intravenous tumour cell inoculation. Animals received intravenous injections with antibody at 2 mg/kg or bortezomib (Velcade) at 0.5 mg/kg on Days 7, 10, 14, and 17 (as indicated by arrows in graph). There were 8 mice per treatment group. Statistical significance was calculated using Log-rank Graphpad Prism software. Significance was determined at *p<0.05, **p<0.01, and ***p<0.001.

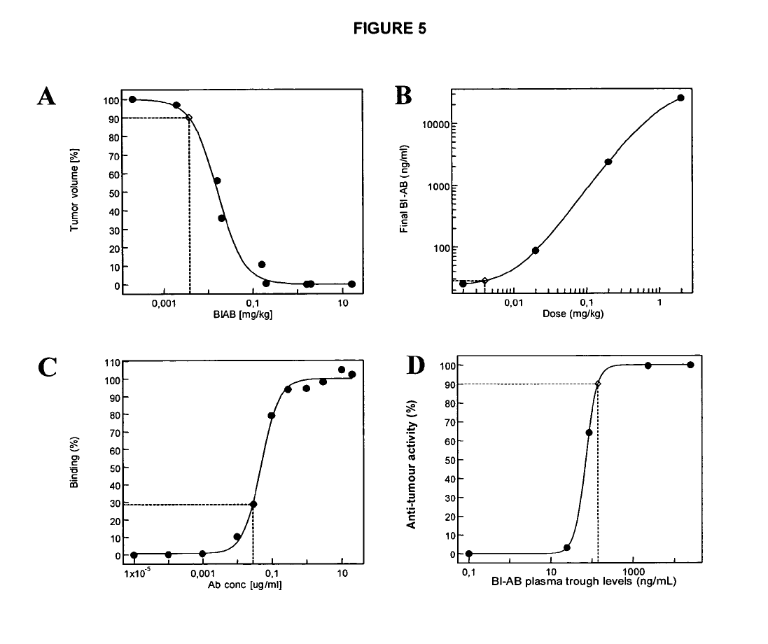

FIG. 5: Calculation of MABEL: determination of BI-AB IC₁₀ value using in vivo pharmacology assay and in vitro correlation with binding.
(A) Tumour cells were injected subcutaneously into the left flank of SCID mice (n=8 per group). Mice received twice-weekly intraperitoneal injections with BI-AB at a range of 20 to 0.002 mg/kg (until control group had to be sacrificed). Graph shows mean IC₁₀ dose for BI-AB inhibition of tumour growth as calculated from two independent experiments each comprising 8 animals per dose group.
(B) Blood samples were collected at different time-points during course of in vivo experimentation and analyzed by ELISA to determine BI-AB trough levels. Graph shows steady state blood levels at IC₁₀ inhibition of tumour growth (28 ng/mL).
(C) BI-AB binds to ARH-77 myeloma cells in a dose-dependent manner. Graph shows percent binding to cells at 28 ng/mL corresponding to blood levels of BI-AB at IC₁₀ inhibition of tumour growth. IC₁₀=concentration at which there is 10% inhibition; ELISA=enzyme-linked immunosorbent assay; MABEL=minimum anticipated biological effect level.
(D) ARH-77myeloma cells were injected subcutaneously into the left flank of SCID mice (n=8 per group). Mice received twice-weekly intravenous injections of BI-AB at 0.002-20 mg/kg until control group had to be sacrificed. Blood samples were collected at termination and analyzed by ELISA to determine BI-AB serum trough levels. Trough BI-AB serum concentrations were plotted as a function of anti-tumor activity and fitted using five parameter log-log curve and XLfit software (IDBS, Guildford, United Kingdom).

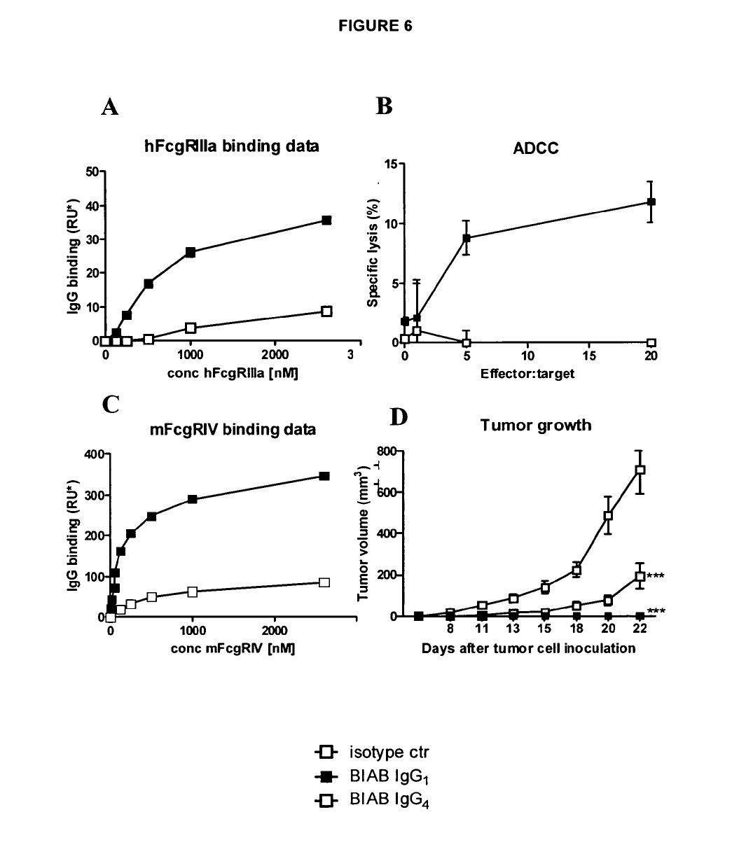

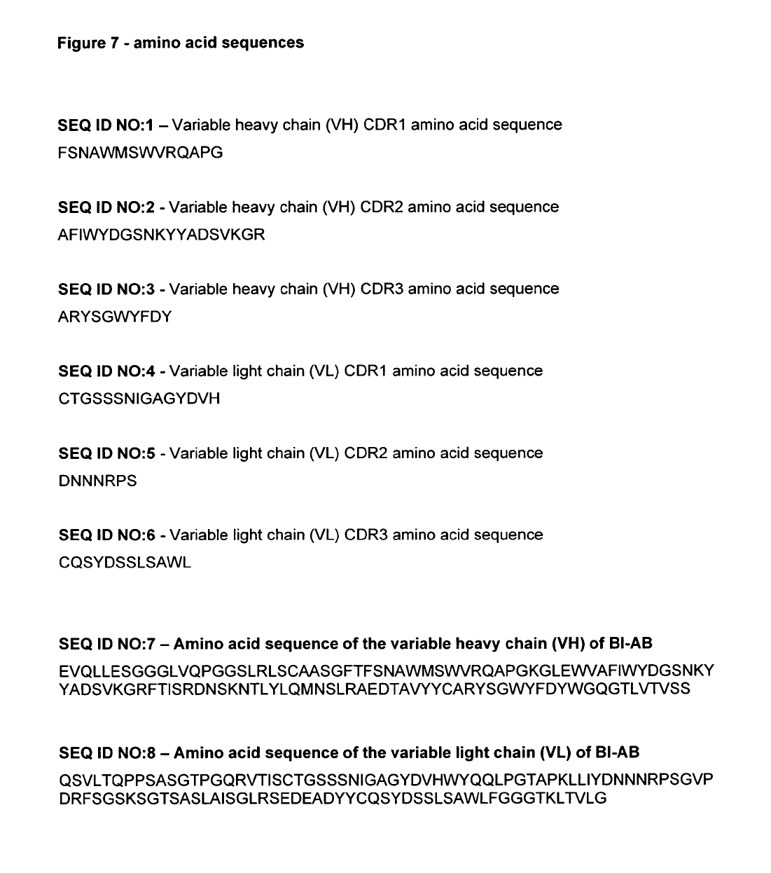

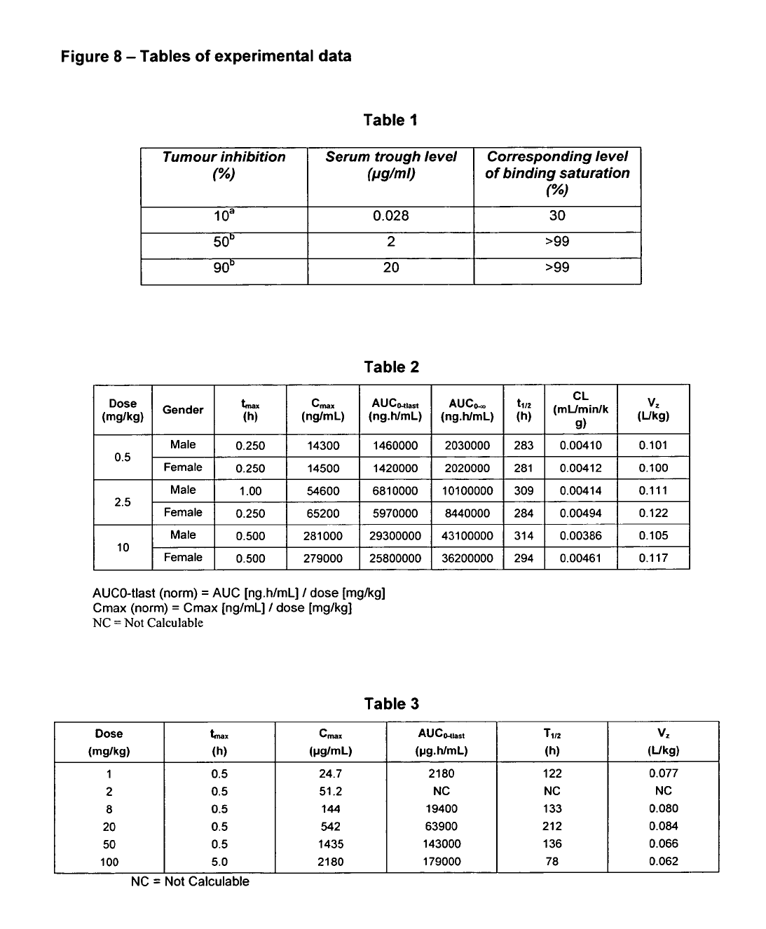

FIG. 6: BI-AB anti-myeloma activity involves both Fc-dependent and Fc-independent mechanisms of action.

Table 1: BI-AB levels associated 10, 50, and 90% anti-tumour activity correlated with levels of receptor binding.
   a—determined in the most sensitive setting in the ARH-77/SCID in vivo tumour model, i.e., early antibody administration.
   b—determined in the advanced ARH-77/SCID in vivo tumour model (mice carrying established tumours)
Table 2: Single dose pharmacokinetics of BI-AB following single IV bolus injection
Table 3: Pharmacokinetics of BI-AB following single escalating IV bolus injection The pharmacokinetics of BI-AB was investigated at dose levels of 1, 2, 8, 20, 50, and 100 mg/kg in the escalation dose toxicity study in cynomolgus monkeys. Samples for plasma analysis were taken after all dose levels. Pharmacokinetic data demonstrated that the systemic exposure increased roughly in proportion to the dose without gender deviations. Cₘₐₓ were generally obtained at the first sampling time of 0.5 hours post-dose. However at 100 mg/kg the Cₘₐₓ was reached 8 hours post-dose. Serum concentrations declined in a generally bi-phasic manner with an elimination time ranging from 69 to 264 hours. The volumes of distribution were similar and showed no dose-dependency. There was some carry-over of the compound between treatments due to the long half-life of BI-AB.

### EXAMPLES

### Example 1

### Administration of the Anti-ICAM-1 Antibody, BI-AB, at Low Doses and at Less Frequent Intervals

### The Antibody

BI-AB is a fully human, high-affinity IgG₁ mAb specific for ICAM-1 that was generated from the proprietary n-CoDeR® human antibody fragment library (BioInvent International AB). BI-AB has been characterized and shown to be potentially suitable for the treatment of multiple myeloma on the basis of its significant and broad anti-myeloma activity in vitro and in vivo. ICAM-1 is a cell-surface protein with broad expression in multiple human malignancies including myeloma and is also upregulated on multiple myeloma cells from patients refractory to chemotherapy (Schmidmaier, 2006). Owing to its cell surface expression, ICAM-1 is highly accessible for antibody-based therapy. Therefore, BI-AB could be an important contribution in treating the growing and clinically important group of patients with refractory and relapsed myeloma.

### Mode of Action

BI-AB binds with high affinity and specificity to ICAM-1 and has significant activity against CD20-positive and CD20-negative multiple myeloma tumours in vivo. In vivo and in vitro mode-of-action studies identify apoptosis and antibody-dependent cell-mediated cytotoxicity (ADCC) as important mechanisms underlying BI-AB anti-tumour activity. In addition, BI-AB binding of soluble intercellular adhesion molecule 1 (sICAM-1) may inhibit tumour angiogenesis and minimize the escape of tumour cells from immunosurveillance.

### Physical and Chemical Properties

BI-AB is a fully human immunoglobulin G subtype 1 (IgG1) monoclonal antibody expressed in Chinese Hamster Ovarian (CHO) cells. The BI-AB antibody has a theoretical molecular weight of approximately 144 kDa based on the predicted amino acid sequence. Mass spectrometry, peptide mapping and SDS-PAGE will determine molecular mass and primary and secondary structures. The biological activity is quantified using an ELISA assay, which quantitatively measures the binding of the BI-AB to ICAM-1.

### Generation of the Antibody

The BI-AB antibody was isolated from BioInvent's proprietary n-CoDeR® human antibody fragment library, converted into a full length, fully human IgG₁ antibody and then transferred to the glutamine synthetase (GS) gene expression system, licensed from Lonza Biologics.

Fetal calf serum from Invitrogen and of USA origin was used during the transfection and selection stages of the cell line development. Serum free adaptation of the cell line was performed before establishment of the RCB.

### In Vivo Studies

BI-AB has significant and broad anti-myeloma activity in vivo, as demonstrated in SCID/xenograft models comprising well-characterized malignant myeloma cell lines representing early and advanced stages of multiple myeloma (FIGS. 2-4). The anti-tumour activity of BI-AB (FIG. 2) has been compared with Rituximab, an anti-CD20 antibody currently in Phase 2 clinical studies for treatment of CD20-positive multiple myeloma, in the SCID/ARH-77 xenograft model of multiple myeloma.

In this model BI-AB was more efficacious and potent compared with Rituximab both in terms of reducing tumour growth and in prolonging survival (FIG. 2B; Kaplan-Meier) in both prophylactic (FIG. 2A and FIG. 2B) and established (FIG. 2C) xenograft models.

Importantly, the significant anti-myeloma activity of BI-AB was not the result of high expression of BI-AB epitopes as compared with expression of Rituximab epitopes. Conversely, multiple myeloma cells expressed significantly more CD20, compared with ICAM-1 as revealed by flow cytometry. Furthermore, immunohistochemical analysis of myeloma tumour tissue harvested at completion of experimentation revealed that BI-AB-treated and Rituximab-treated animals retained both ICAM-1 and CD20 expression throughout the course of experimentation

In addition to the significant in vivo anti-tumour effects observed when using the SCID/ARH-77 xenograft model, BI-AB showed significant anti-myeloma activity also in CD20-negative ICAM-1-positive myeloma cell lines (FIG. 3A-C). BI-AB did not affect tumour growth of the ICAM-1-negative myeloma cell line OPM-2 (FIG. 3D), demonstrating that anti-myeloma activity was ICAM-1 dependent.

BI-AB anti-myeloma activity has additionally been investigated in a disseminated tumour model (a well-established experimental model of human multiple myeloma) at an independent contract research organization. BI-AB significantly prolonged survival compared with control-treated mice when antibody treatment commenced 1 day (data not shown) or 7 days after intravenous tumour cell inoculation (FIG. 4). In addition, BI-AB was benchmarked against Bortezomib and was shown to be superior. After 190 days, when the experiment was terminated, 25% of BI-AB-treated mice were still alive. Control-treated mice had a mean survival time of 35 days and had all died by Day 39.

In addition to myeloma xenograft models, BI-AB also confers significant anti-lymphoma activity as demonstrated in prophylactic and established SCID/B-cell lymphoma tumour xenograft models (data not shown).

### MABEL (Minimal Anticipated Biological Effect Level)

To identify an appropriate starting dose for the initial clinical study we determined MABEL in (1) the ARH-77/SCID in vivo tumour model and (2) receptor occupancy in vitro using human tumour cells.

In the ARH-77/SCID in vivo tumour model BI-AB levels associated with anti-tumour activity have been identified and are used as a basis for predicting where a minimal response could be anticipated in humans. In this model, using the most sensitive setting (i.e. early antibody administration) MABEL is defined as the BI-AB serum trough concentration at which there is 10% inhibition (IC₁₀) of tumour growth. The dose response curve of BI-AB in this model is sigmoidal, and no indications of u-shaped or bell-shaped response curves have been observed (FIG. 5). The MABEL/IC₁₀ dose in this model was determined to be 0.004 mg/kg when using a twice-weekly dosing regimen (FIG. 5A), and corresponding to a steady state serum concentration of 28 ng/mL BI-AB as described below.

Mean BI-AB serum trough concentrations of different mouse dose groups were determined by ELISA analysis of blood samples collected at different time-points during the course of in vivo experimentation. Trough BI-AB serum concentrations were plotted as a function of dose and were fitted using five parameter log-log curve and XLfit software (IDBS, Guildford, United Kingdom) (FIG. 5B).

Receptor occupancy has been examined in vitro using human tumour cells and the BI-AB level corresponding to binding saturation has been identified. It is assumed that at this concentration, ICAM-1 epitopes on cells of the vascular compartment should be saturated, and any side effects related to the physiological function of ICAM-1 are expected to be near their peak. Similarly, at 30% binding saturation, any ICAM-1 dependent biological responses, including those associated with side effects, are expected to be triggered. The BI-AB levels associated 10, 50, and 90% anti-tumour activity, respectively have also been correlated with the levels of receptor binding saturation and are presented in Table 1.

These results are used in the identification of an appropriate starting dose and the use of this together with a slow and long infusion to obtain a safe starting dose regimen is described in the clinical study protocol.

By applying the equation used to fit the data, a trough BI-AB serum concentration of 28 ng/mL was found to correspond to the 0.004 mg/kg dose and 10% inhibition of tumour growth (FIG. 5B). At this concentration approximately 30% of ARH-77 ICAM-1 epitopes were occupied by antibody in vitro (FIG. 5C).

### Pharmacokinetics in the Rat

A pharmacokinetic (PK) study of BI-AB in the rat has been performed. In this study, the doses of the antibody tested were in such excess in relation to the target antigen concentration that any binding to the target antigen would not be of significant importance for the PK profile. This fact serves as the rationale for performing a PK study in a species where the antibody will not bind to the target antigen. The pharmacokinetics of BI-AB in the rat has been investigated following a single IV bolus injection at dose levels of 0.5, 2.5 and 10 mg/kg. The pharmacokinetics demonstrated that the systemic exposure of BI-AB increased roughly in proportion to dose. Maximum serum concentrations were generally obtained within the first hour. Serum concentrations of BI-AB declined in a generally multi-phasic manner with an elimination half-life ranging from 281 to 314 hours. The clearance and volumes of distribution (V_{z} and Vₛₛ) appeared to be dose independent. The systemic exposure of BI-AB appeared to be generally independent of sex. The single dose pharmacokinetics are shown in Table 2.

### Pharmacokinetics in the Cynomolgus Monkey

The pharmacokinetics of BI-AB was investigated at dose levels of 1, 2, 8, 20, 50, and 100 mg/kg in the escalation dose toxicity study in cynomolgus monkeys. Samples for plasma analysis were taken after all dose levels.

Pharmacokinetic data demonstrated that the systemic exposure increased roughly in proportion to the dose without gender deviations. Cₘₐₓ were generally obtained at the first sampling time of 0.5 hours post-dose. However at 100 mg/kg the Cₘₐₓ was reached 8 hours post-dose. Serum concentrations declined in a generally bi-phasic manner with an elimination time ranging from 69 to 264 hours. The volumes of distribution were similar and showed no dose-dependency. There was some carry-over of the compound between treatments due to the long half-life of BI-AB.

The systemic exposures of BI-AB are shown in Table 3.

### REFERENCES

Aalinkeel R, Nair M P, Sufrin G, Mahajan S D, Chadha K C, Chawda R P, et al. Gene expression of angiogenic factors correlates with metastatic potential of prostate cancer cells. Cancer Res. 2004. 64(15): 5311-21.
Clynes R A, Towers T L, Presta L G, Ravetch J V. Inhibitory Fc receptors modulate in vivo cytoxicity against tumour targets. Nat. Med. 2000. 6: 2738-2743.
A. Dowlati, R. Gray, A. B. Sandler et al., Clin Cancer Res 14 (5), 1407 (2008).
A. J. Gearing and W. Newman, Immunol Today 14 (10), 506 (1993); P. Sanchez-Rovira, E. Jimenez, J. Carracedo et al., Eur J Cancer 34 (3), 394 (1998).
Y. S. Gho, H. K. Kleinman, and G. Sosne, Cancer Res 59 (20), 5128 (1999); Y. S. Gho, P. N. Kim, H. C. Li et al., Cancer Res 61 (10), 4253 (2001).
A. Grothey, P. Heistermann, S. Philippou et al., Br J Cancer 77 (5), 801 (1998).
T. Hideshima, C. Mitsiades, G. Tonon et al., Nat Rev Cancer 7 (8), 585 (2007).
Huang Y W, Burrows F J, Vitetta E S. Cytotoxicity of a novel anti-ICAM-1 immunotoxin on human myeloma cell lines. Hybridoma. 1993. 12(6): 661-675.
Huang Y W, Richardson J A, Vitetta E S. Anti-CD54 (ICAM-1) has antitumor activity in SCID mice with human myeloma cells. Cancer Res. 1995. 55(3): 610-616.
C. Huang and T. A. Springer, J Biol Chem 270 (32), 19008 (1995); Y. W. Huang, J. A. Richardson, A. W. Tong et al., Cancer Res 53 (6), 1392 (1993).
W. C. Huang, S. T. Chan, T. L. Yang et al., Carcinogenesis 25 (10), 1925 (2004).
E. Horst, T. Radaszkiewicz, A. Hooftman-den Otter et al., Leukemia 5 (10), 848 (1991).
J. P. Johnson, B. G. Stade, U. Hupke et al., Immunobiology 178 (3), 275 (1988).
Y. S. Kim, G. B. Park, H. K. Song et al., J Immunother (1997) 30 (7), 727 (2007).
Kyle R A, Rajkumar S V. Blood. 2008 Mar. 15; 111(6):2962-72. Review.
Kyle R A, Rajkumar S V. N Engl J. Med. 2004 Oct. 28; 351(18):1860-73. Review
Y. Maruo, A. Gochi, A. Kaihara et al., Int J Cancer 100 (4), 486 (2002).
M. E. Miele, C. F. Bennett, B. E. Miller et al., Exp Cell Res 214 (1), 231 (1994).
Ravikumar Aalinkeel, Madhavan P. N. Nair, Gerald Sufrin et al., Cancer Res 64 (15), 5311 (2004).
Y. Roche, D. Pasquier, J. J. Rambeaud et al., Thromb Haemost 89 (6), 1089 (2003).
S. Rose-John and P. C. Heinrich, Biochem J 300 (Pt 2), 281 (1994).
C. Rosette, R. B. Roth, P. Oeth et al., Carcinogenesis 26 (5), 943 (2005).
R. Schmidmaier, K. Morsdorf, P. Baumann et al., Int J Biol Markers 21 (4), 218 (2006).
Smallshaw J E, Coleman E, Spiridon C, Vitetta E S. The generation and anti-myeloma activity of a chimeric anti-CD54 antibody, cUV3. J Immunother (1997). 2004. 27(6): 419-424.
A. van de Stolpe and P. T. van der Saag, J Mol Med 74 (1), 13 (1996); K. Nasu, H. Narahara, Y. Etoh et al., Gynecol Oncol 65 (2), 304 (1997); M. J. Terol, M. Tormo, J. A. Martinez-Climent et al., Ann Oncol 14 (3), 467 (2003).
S. Wang, E. J. Coleman, L. M. Pop et al., Int J Cancer 27 (6), 419 (2005).
Wang S, Coleman E J, Pop L M, Brooks K J, Vitetta E S, Niederkorn J Y. Effect of an anti-CD54 (ICAM-1) monoclonal antibody (UV3) on the growth of human uveal melanoma cells transplanted heterotopically and orthotopically in SCID mice. Int J. Cancer. 2006. 118(4): 932-941.
Weng W, Levy R. Two immunoglobulin G fragment C receptor polymorphisms independently predict response to Rituximab in patients with follicular lymphoma. Journal of Clinical Oncology. 2003. 21(21): 3940-3947.

### Example 2

### Exemplary Pharmaceutical Formulations

Whilst it is possible for an antibody, an antigen-binding fragment, fusion, variant or derivative of the invention to be administered alone, it is preferable to present it as a medicament or pharmaceutical formulation, together with one or more acceptable carriers. The carrier(s) must be “acceptable” in the sense of being compatible with the agent of the invention and not deleterious to the recipients thereof. Typically, the carriers will be water or saline which will be sterile and pyrogen-free.

The following examples illustrate medicaments and pharmaceutical compositions according to the invention in which the active ingredient is an antibody, an antigen-binding fragment, fusion, variant or derivative of the invention.

### Example 2A

### Injectable Formulation

| | | |
|---|---|---|
| Active ingredient | 10 | mg |
| Sterile, pyrogen free phosphate buffer (pH7.0) | to 1 | ml |

The active ingredient is dissolved in most of the phosphate buffer (35-40° C.), then made up to volume and filtered through a sterile micropore filter into a sterile 10 ml amber glass vial (type 1) and sealed with sterile closures and overseals.

### Example 2B

### Intramuscular Injection

| | | |
|---|---|---|
| Active ingredient | 10 | mg |
| Benzyl Alcohol | 0.10 | g |
| Glucofurol 75 ® | 1.45 | g |
| Water for Injection | q.s. to 1.00 | ml |

The active ingredient is dissolved in the glycofurol. The benzyl alcohol is then added and dissolved, and water added to 3 ml. The mixture is then filtered through a sterile micropore filter and sealed in sterile 3 ml glass vials (type 1).

### Example 2C

### Subcutaneous Injection

| BI-AB (i.e. active ingredient) | 10 | mg |
|---|---|---|
| Sodium chloride (i.e. buffer) | 8.77 | mg |
| Sodiumacetate-3-hydrate (i.e. buffer) | 2.35 | mg |
| Acetic acid (i.e. buffer) | 0.16 | μL |
| Hydrochloric acid (i.e. pH adjustor) | to pH 5.5 | |
| Sodium hydroxide (i.e. pH adjustor) | to pH 5.5 | |
| Polysorbate 20 (i.e. surfactant) | 0.5 | mg |
| Water for Injection (i.e. pH adjustor) | to 1 | ml |

### Example 3

### Treatment of Multiple Myeloma in a Patient Using a Medicament or Pharmaceutical Composition of the Invention

A patient presenting with multiple myeloma may be identified by clinical examination.

Because many organs can be affected by myeloma, the symptoms and signs vary greatly. A mnemonic sometimes used to remember the common tetrad of multiple myeloma is CRAB: C=Calcium (elevated), R=Renal failure, A=Anemia, B=Bone lesions. Myeloma has many possible symptoms including:

### Bone Pain

Myeloma bone pain usually involves the spine and ribs, and worsens with activity. Persistent localized pain may indicate a pathological bone fracture. Involvement of the vertebrae may lead to spinal cord compression. Myeloma bone disease is due to proliferation of tumor cells and release of Interleukin 1, IL-1, also known as osteoclast activating factor (OAF), which stimulates osteoclasts to break down bone. These bone lesions are lytic in nature and are best seen in plain radiographs, which may show “punched-out” resorptive lesions. The breakdown of bone also leads to release of calcium into the blood, leading to hypercalcemia and its associated symptoms.

### Infection

The most common infections are pneumonias and pyelonephritis. Common pneumonia pathogens include S. pneumoniae, S. aureus, and K. pneumoniae, while common pathogens causing pyelonephritis include E. coli and other gram-negative organisms. The greatest risk period for the occurrence of infection is in the initial few months after the start of chemotherapy. The increased risk of infection is due to immune deficiency resulting from diffuse hypogammaglobulinemia, which is due to decreased production and increased destruction of normal antibodies.

### Renal Failure

Renal failure may develop both acutely and chronically. It is commonly due to hypercalcemia (see above). It may also be due to tubular damage from excretion of light chains, also called Bence Jones proteins, which can manifest as the Fanconi syndrome (type II renal tubular acidosis). Other causes include glomerular deposition of amyloid, hyperuricemia, recurrent infections (pyelonephritis), and local infiltration of tumor cells.

### Anemia

The anemia found in myeloma is usually normocytic and normochromic. It results from the replacement of normal bone marrow by infiltrating tumor cells and inhibition of normal red blood cell production (hematopoiesis) by cytokines.

### Neurological Symptoms

Common problems are weakness, confusion and fatigue due to hypercalcemia. Headache, visual changes and retinopathy may be the result of hyperviscosity of the blood depending on the properties of the paraprotein. Finally, there may be radicular pain, loss of bowel or bladder control (due to involvement of spinal cord leading to cord compression) or carpal tunnel syndrome and other neuropathies (due to infiltration of peripheral nerves by amyloid). It may give rise to paraplegia in late presenting cases.

The typical clinical presentation of myeloma is a patient with severe pain caused by pathological bone fractures, particularly in the rib cage or vertebral column. Other common features include renal failure and hypercalcemia, and bone marrow insufficiency with anemia and thrombocytopenia is also common. Organ failure is sometimes caused, and typically involves heart and kidneys, resulting in severe cardiac arrhythmias or failure and nephrotic syndrome, respectively. Bleeding diathesis is also common in amyloid light-chain amyloidosis.

Clinical manifestations of multiple myeloma result from the replacement of normal marrow (containing stem cells, red and white blood cells, platelets, and plasma) with malignant plasma cells. Signs and symptoms include bone destruction, pain, fever, weight loss, anaemia, repeated infections, bleeding disorders, hypercalcaemia, proteinaemia, and renal failure (caused by excessive amounts of M protein in blood and urine).

The diagnosis of multiple myeloma involves a bone marrow biopsy, blood test, and x-rays, as are known in the art. A confirmed multiple myeloma diagnosis usually requires that the following criteria are met: (i) greater than 10% plasma cells in bone marrow; (ii) M protein in blood or urine; (iii) the presence of lytic bone lesions, and (iv) the presence of soft tissue plasmacytomas (National Cancer Institute website, accessed February 2008).

The following medicament is administered to the patient subcutaneously, according to the methods and uses of the invention:

| BI-AB (i.e. active ingredient) | 10 | mg |
|---|---|---|
| Sodium chloride (i.e. buffer) | 8.77 | mg |
| Sodiumacetate-3-hydrate (i.e. buffer) | 2.35 | mg |
| Acetic acid (i.e. buffer) | 0.16 | μL |
| Hydrochloric acid (i.e. pH adjustor) | to pH 5.5 | |
| Sodium hydroxide (i.e. pH adjustor) | to pH 5.5 | |
| Water for Injection (i.e. pH adjustor) | to 1 | ml |
| Polysorbate 20 (i.e. surfactant) | 0.5 | mg |

Treatment using the above medicament and administration regimen is continued for an appropriate period (for example, over months or years) until the presenting symptoms disappear.

1. (canceled)

2. An antibody or an antigen-binding fragment thereof with binding specificity for ICAM-1,
or a variant, fusion or derivative of said antibody or an antigen-binding fragment, or a fusion of a said variant or derivative thereof, with binding specificity for ICAM-1,
wherein the antibody or antigen-binding fragment thereof comprises one or more of the following amino acid sequences:
[SEQ ID NO: 1]
FSNAWMSWVRQAPG; and/or
[SEQ ID NO: 2]
AFIWYDGSNKYYADSVKGR; and/or
[SEQ ID NO: 3]
ARYSGWYFDY; and/or
[SEQ ID NO: 4]
CTGSSSNIGAGYDVH; and/or
[SEQ ID NO: 5]
DNNNRPS; and/or
[SEQ ID NO: 6]
CQSYDSSLSAWL.
for use in the treatment of cancer, the treatment comprising the step of administering to a patient in need thereof an effective amount of the antibody, antigen-binding fragment, variant, fusion or derivative thereof.

3. A method for treating cancer in an individual, the method comprising the step of administering to a patient in need thereof an effective amount of:
an antibody or an antigen-binding fragment thereof with binding specificity for ICAM-1,
or a variant, fusion or derivative of said antibody or an antigen-binding fragment, or a fusion of a said variant or derivative thereof, with binding specificity for ICAM-1,
wherein the antibody or antigen-binding fragment thereof comprises one or more of the following amino acid sequences:
[SEQ ID NO: 1]
FSNAWMSWVRQAPG; and/or
[SEQ ID NO: 2]
AFIWYDGSNKYYADSVKGR; and/or
[SEQ ID NO: 3]
ARYSGWYFDY; and/or
[SEQ ID NO: 4]
CTGSSSNIGAGYDVH; and/or
[SEQ ID NO: 5]
DNNNRPS; and/or
[SEQ ID NO: 6]
CQSYDSSLSAWL.

4. The method according to claim 3 wherein the effective amount of the antibody, antigen-binding fragment, variant, fusion or derivative thereof is between about 0.02 mg/kg to 2 mg/kg of the antibody, antigen-binding fragment, variant, fusion or derivative thereof.

5. The method according to claim 4 wherein the effective amount of the antibody, antigen-binding fragment, variant, fusion or derivative thereof is selected from:
between 0.02 mg/kg and 0.03 mg/kg; or
between 0.02 mg/kg and 0.30 mg/kg; or
between 0.02 mg/kg and 0.10 mg/kg; or
between 0.10 mg/kg and 0.30 mg/kg.

6. The method according to claim 5 wherein the effective amount of the antibody, antigen-binding fragment, variant, fusion or derivative thereof is selected from:
0.016 mg/kg; or
0.02 mg/kg; or
0.03 mg/kg; or
0.10 mg/kg; or
0.16 mg/kg; or
0.30 mg/kg.

7.-8. (canceled)

9. A method for treating cancer in an individual, the method comprising the step of administering to a patient in need thereof a single dosage at a frequency of once per week or less of an effective amount of:
an antibody or an antigen-binding fragment thereof with binding specificity for ICAM-1,
or a variant, fusion or derivative of said antibody or an antigen-binding fragment, or a fusion of a said variant or derivative thereof with binding specificity for ICAM-1,
wherein the antibody or antigen-binding fragment thereof comprises one or more of the following amino acid sequences:
[SEQ ID NO: 1]
FSNAWMSWVRQAPG; and/or
[SEQ ID NO: 2]
AFIWYDGSNKYYADSVKGR; and/or
[SEQ ID NO: 3]
ARYSGWYFDY; and/or
[SEQ ID NO: 4]
CTGSSSNIGAGYDVH; and/or
[SEQ ID NO: 5]
DNNNRPS; and/or
[SEQ ID NO: 6]
CQSYDSSLSAWL.

10. The method according to claim 9 wherein the effective amount of the antibody, antigen-binding fragment, variant, fusion or derivative thereof is administered in a single dosage at a frequency selected from:
once in eight days; or
once in nine days; or
once in ten days; or
once in eleven days; or
once in twelve days; or
once in thirteen days; or
once in fourteen days; or
once in twenty-one days.

11.-12. (canceled)

13. The method of claim 9, wherein the effective amount of the antibody, antigen-binding fragment, variant, fusion or derivative thereof is between about 0.02 mg/kg to 2 mg/kg of the antibody, antigen-binding fragment, variant, fusion or derivative thereof.

14. The method according to claim 13 wherein the effective amount of the antibody, antigen-binding fragment, variant, fusion or derivative thereof and administration frequency is selected from:
between 0.02 mg/kg and 0.03 mg/kg, administered at a frequency of once in seven days; or
between 0.02 mg/kg and 0.03 mg/kg, administered at a frequency of once in eight days; or
between 0.02 mg/kg and 0.03 mg/kg, administered at a frequency of once in nine days; or
between 0.02 mg/kg and 0.03 mg/kg, administered at a frequency of once in ten days; or
between 0.02 mg/kg and 0.03 mg/kg, administered at a frequency of once in eleven days; or
between 0.02 mg/kg and 0.03 mg/kg, administered at a frequency of once in twelve days; or
between 0.02 mg/kg and 0.03 mg/kg, administered at a frequency of once in fourteen days; or
between 0.02 mg/kg and 0.03 mg/kg, administered at a frequency of once in twenty-one days.

15. The method according to claim 13 wherein the effective amount of the antibody, antigen-binding fragment, variant, fusion or derivative thereof and administration frequency is selected from:
between 0.02 mg/kg and 0.30 mg/kg, administered at a frequency of once in seven days; or
between 0.02 mg/kg and 0.30 mg/kg, administered at a frequency of once in eight days; or
between 0.02 mg/kg and 0.30 mg/kg, administered at a frequency of once in nine days; or
between 0.02 mg/kg and 0.30 mg/kg, administered at a frequency of once in ten days; or
between 0.02 mg/kg and 0.30 mg/kg, administered at a frequency of once in eleven days; or
between 0.02 mg/kg and 0.30 mg/kg, administered at a frequency of once in twelve days; or
between 0.02 mg/kg and 0.30 mg/kg, administered at a frequency of once in fourteen days; or
between 0.02 mg/kg and 0.30 mg/kg, administered at a frequency of once in twenty-one days.

16. The method according to claim 13 wherein the effective amount of the antibody, antigen-binding fragment, variant, fusion or derivative thereof and administration frequency is selected from:
between 0.02 mg/kg and 0.10 mg/kg, administered at a frequency of once in seven days; or
between 0.02 mg/kg and 0.10 mg/kg, administered at a frequency of once in eight days; or
between 0.02 mg/kg and 0.10 mg/kg, administered at a frequency of once in nine days; or
between 0.02 mg/kg and 0.10 mg/kg, administered at a frequency of once in ten days; or
between 0.02 mg/kg and 0.10 mg/kg, administered at a frequency of once in eleven days; or
between 0.02 mg/kg and 0.10 mg/kg, administered at a frequency of once in twelve days; or
between 0.02 mg/kg and 0.10 mg/kg, administered at a frequency of once in fourteen days; or
between 0.02 mg/kg and 0.10 mg/kg, administered at a frequency of once in twenty-one days.

17. The method according to claim 13 wherein the effective amount of the antibody, antigen-binding fragment, variant, fusion or derivative thereof and administration frequency is selected from:
between 0.10 mg/kg and 0.30 mg/kg, administered at a frequency of once in seven days; or
between 0.10 mg/kg and 0.30 mg/kg, administered at a frequency of once in eight days; or
between 0.10 mg/kg and 0.30 mg/kg, administered at a frequency of once in nine days; or
between 0.10 mg/kg and 0.30 mg/kg, administered at a frequency of once in ten days; or
between 0.10 mg/kg and 0.30 mg/kg, administered at a frequency of once in eleven days; or
between 0.10 mg/kg and 0.30 mg/kg, administered at a frequency of once in twelve days; or
between 0.10 mg/kg and 0.30 mg/kg, administered at a frequency of once in fourteen days; or
between 0.10 mg/kg and 0.30 mg/kg, administered at a frequency of once in twenty-one days.

18. The method according to claim 13 wherein the effective amount of the antibody, antigen-binding fragment, variant, fusion or derivative thereof and administration frequency is selected from:
0.016 mg/kg, administered at a frequency of once in seven days; or
0.016 mg/kg, administered at a frequency of once in eight days; or
0.016 mg/kg, administered at a frequency of once in nine days; or
0.016 mg/kg, administered at a frequency of once in ten days; or
0.016 mg/kg, administered at a frequency of once in eleven days; or
0.016 mg/kg, administered at a frequency of once in twelve days; or
0.016 mg/kg, administered at a frequency of once in fourteen days; or
0.016 mg/kg, administered at a frequency of once in twenty-one days.

19. The method according to claim 13 wherein the effective amount of the antibody, antigen-binding fragment, variant, fusion or derivative thereof and administration frequency is selected from:
0.02 mg/kg, administered at a frequency of once in seven days; or
0.02 mg/kg, administered at a frequency of once in eight days; or
0.02 mg/kg, administered at a frequency of once in nine days; or
0.02 mg/kg, administered at a frequency of once in ten days; or
0.02 mg/kg, administered at a frequency of once in eleven days; or
0.02 mg/kg, administered at a frequency of once in twelve days; or
0.02 mg/kg, administered at a frequency of once in fourteen days; or
0.02 mg/kg, administered at a frequency of once in twenty-one days.

20. The method according to claim 13 wherein the effective amount of the antibody, antigen-binding fragment, variant, fusion or derivative thereof and administration frequency is selected from:
0.03 mg/kg, administered at a frequency of once in seven days; or
0.03 mg/kg, administered at a frequency of once in eight days; or
0.03 mg/kg, administered at a frequency of once in nine days; or
0.03 mg/kg, administered at a frequency of once in ten days; or
0.03 mg/kg, administered at a frequency of once in eleven days; or
0.03 mg/kg, administered at a frequency of once in twelve days; or
0.03 mg/kg, administered at a frequency of once in fourteen days; or
0.03 mg/kg, administered at a frequency of once in twenty-one days.

21. The method according to claim 13 wherein the effective amount of the antibody, antigen-binding fragment, variant, fusion or derivative thereof and administration frequency is selected from:
0.10 mg/kg, administered at a frequency of once in seven days; or
0.10 mg/kg, administered at a frequency of once in eight days; or
0.10 mg/kg, administered at a frequency of once in nine days; or
0.10 mg/kg, administered at a frequency of once in ten days; or
0.10 mg/kg, administered at a frequency of once in eleven days; or
0.10 mg/kg, administered at a frequency of once in twelve days; or
0.10 mg/kg, administered at a frequency of once in fourteen days; or
0.10 mg/kg, administered at a frequency of once in twenty-one days.

22. The method according to claim 13 wherein the effective amount of the antibody, antigen-binding fragment, variant, fusion or derivative thereof and administration frequency is selected from:
0.30 mg/kg, administered at a frequency of once in seven days; or
0.30 mg/kg, administered at a frequency of once in eight days; or
0.30 mg/kg, administered at a frequency of once in nine days; or
0.30 mg/kg, administered at a frequency of once in ten days; or
0.30 mg/kg, administered at a frequency of once in eleven days; or
0.30 mg/kg, administered at a frequency of once in twelve days; or
0.30 mg/kg, administered at a frequency of once in fourteen days; or
0.30 mg/kg, administered at a frequency of once in twenty-one days.

23. The method according to claim 3 or 9 wherein ICAM-1 is localised on the surface of a cell.

24. The method according to claim 23 wherein the cell is a cancer cell.

25. The method according to claim 24 wherein the cancer cell is selected from the group consisting of: a myeloma cell; a melanoma cell; a lung cancer cell; a gastric cancer cell; a bladder cancer cell; a breast cancer cell; a prostate cancer cell; a lymphoma cell; a renal cancer cell; a hepatocellular carcinoma cell.

26. The method according to claim 24 wherein the cancer cell is a myeloma cell.

27. The method according to claim 3 or 9, wherein the antibody or antigen-binding fragment, or a variant, fusion or derivative thereof, comprises or consists of an intact antibody.

28. The method according to claim 3 or 9, wherein the antibody or antigen-binding fragment, or a variant, fusion or derivative thereof, comprises or consists of an antigen-binding fragment selected from the group consisting of: an Fv fragment; an Fab fragment; an Fab-like fragment.

29. The method according to claim 28 wherein the Fv fragment is a single chain Fv fragment or a disulphide-bonded Fv fragment.

30. The method according to claim 28 wherein the Fab-like fragment is an Fab′ fragment or an F(ab)₂ fragment.

31. The method according to claim 3 or 9, wherein the antibody is a recombinant antibody.

32. The method according to claim 3 or 9, wherein the antibody is a monoclonal antibody.

33. The method according to claim 3 or 9, wherein the antibody or antigen-binding fragment thereof is a human antibody or humanised antibody.

34. The method according to claim 3 or 9, wherein the antibody, fragment, variant, fusion or derivative comprises the amino acid sequences of SEQ ID NOS: 1 to 6.

35. The method according to claim 3 or 9, wherein the heavy chain variable region of the antibody, fragment, variant, fusion or derivative comprises the following CDRs:
[SEQ ID NO: 1]
FSNAWMSWVRQAPG;
and
[SEQ ID NO: 2]
AFIWYDGSNKYYADSVKGR;
and
[SEQ ID NO: 3]
ARYSGWYFDY.

36. The method according to claim 34 wherein the heavy chain variable region of the antibody, fragment, variant, fusion or derivative comprises the amino acid sequence of SEQ ID NO: 7.

37. The method according to claim 3 or 9, wherein the light chain variable region of the antibody, fragment, variant, fusion or derivative comprises the following CDRs:
[SEQ ID NO: 4]
CTGSSSNIGAGYDVH;
and
[SEQ ID NO: 5]
DNNNRPS;
and
[SEQ ID NO: 6]
CQSYDSSLSAWL.

38. The method according to claim 37 wherein the light chain variable region of the antibody, fragment, variant, fusion or derivative comprises the amino acid sequence of SEQ ID NO: 8.

39. The method according to claim 3 or 9, wherein the antibody or antigen-binding fragment, variant, fusion or derivative comprises a heavy chain variable region comprising the following CDRs:
[SEQ ID NO: 1]
FSNAWMSWVRQAPG;
and
[SEQ ID NO: 2]
AFIWYDGSNKYYADSVKGR;
and
[SEQ ID NO: 3]
ARYSGWYFDY;
and a light chain variable region comprising the following CDRs:
[SEQ ID NO: 4]
CTGSSSNIGAGYDVH;
and
[SEQ ID NO: 5]
DNNNRPS;
and
[SEQ ID NO: 6]
CQSYDSSLSAWL.

40. The method according to claim 3 or 9, wherein the antibody or antigen-binding fragment, variant, fusion or derivative comprises a heavy chain variable region having the amino acid sequence of SEQ ID NO:7 and a light chain variable region having the amino acid sequence of SEQ ID NO:8.

41. The method according to claim 3 or 9, wherein the antibody or antigen-binding fragment, or variant, fusion or derivative thereof, is capable of specifically binding ICAM-1 localised on the surface of a cell and inhibiting and/or preventing proliferation of that cell.

42. The method according to claim 3 or 9, wherein the antibody or antigen-binding fragment, or variant, fusion or derivative thereof, is capable of specifically binding ICAM-1 localised on the surface of a cell and inducing apoptosis of that cell.

43. The method according to claim 3 or 9, wherein the antibody or antigen-binding fragment, or variant, fusion or derivative thereof, is capable of specifically binding ICAM-1 localised on the surface of a cell and inducing antibody-dependent cell cytotoxicity against that cell.

44. The method according to claim 3 or 9, wherein the antibody or antigen-binding fragment has efficacy in the treatment of cancer.

45. The method according to claim 44, wherein the cancer is selected from the group consisting of multiple myeloma; melanoma; lung cancer; gastric cancer; bladder cancer; breast cancer; prostate cancer; lymphoma; renal cancer; hepatocellular carcinoma.

46. The method according to claim 45 wherein the cancer is multiple myeloma.

47.-48. (canceled)


SEQUENCE LISTING (8 sequences)

SEQ ID NO: 1 - protein, 14 aa, Artificial Sequence
  BI-AB variable heavy chain (VH) CDR1 amino acid sequence
FSNAWMSWVRQAPG

SEQ ID NO: 2 - protein, 19 aa, Artificial Sequence
  BI-AB variable heavy chain (VH) CDR2 amino acid sequence
AFIWYDGSNKYYADSVKGR

SEQ ID NO: 3 - protein, 10 aa, Artificial Sequence
  BI-AB variable heavy chain (VH) CDR3 amino acid sequence
ARYSGWYFDY

SEQ ID NO: 4 - protein, 15 aa, Artificial Sequence
  BI-AB variable light chain (VL) CDR1 amino acid sequence
CTGSSSNIGAGYDVH

SEQ ID NO: 5 - protein, 7 aa, Artificial Sequence
  BI-AB variable light chain (VL) CDR2 amino acid sequence
DNNNRPS

SEQ ID NO: 6 - protein, 12 aa, Artificial Sequence
  BI-AB variable light chain (VL) CDR3 amino acid sequence
CQSYDSSLSAWL

SEQ ID NO: 7 - protein, 117 aa, Artificial Sequence
  BI-AB variable heavy chain (VH) amino acid sequence
EVQLLESGGGLVQPGGSLRLSCAASGFTFSNAWMSWVRQAPGKGLEWVAFIWYDGSNKYYADSVKGRFTISRDNSKNTLYLQMNSLRAEDTAVYYCARYSGWYFDYWGQGTLVTVSS

SEQ ID NO: 8 - protein, 112 aa, Artificial Sequence
  BI-AB variable light chain (VL) amino acid sequence
QSVLTQPPSASGTPGQRVTISCTGSSSNIGAGYDVHWYQQLPGTAPKLLIYDNNNRPSGVPDRFSGSKSGTSASLAISGLRSEDEADYYCQSYDSSLSAWLFGGGTKLTVLG